(19) United States
(12) Patent Application Publication
(10) Pub. No.: US 2023/0390036 A1
(43) Pub. Date: Dec. 7, 2023

(54) AUTO-DENTURE DESIGN SETUP SYSTEMS

(71) Applicant: VOYAGER DENTAL, INC., EDEN PRAIRIE, MN (US)

(72) Inventors: Michael C. Marshall, Edina, MN (US); John Madden, Minneapolis, MN (US); Stephen B. Siegfried Floe, Chanhassen, MN (US)

(21) Appl. No.: 18/328,584

(22) Filed: Jun. 2, 2023

Related U.S. Application Data
(60) Provisional application No. 63/348,217, filed on Jun. 2, 2022.

(51) Int. Cl. A61C 13/34; A61C 7/00

(52) U.S. Cl. CPC A61C 13/34; A61C 7/002; A61C 2007/004

(57) ABSTRACT

Disclosed are systems and methods for performing an auto-setup of a digital denture model. A method includes accessing, by a computer system, a digital denture model for a patient including upper and lower teeth, defining an arch form for the lower teeth, defining an occlusal plane relative to the arch form, identifying datums for each tooth in the digital denture model, leveling each tooth in the digital denture model based on the respective datums being positioned relative to the occlusal plane, and snapping each tooth in the digital denture model to the arch form. Until a threshold level of movement is achieved between each tooth and at least one of (i) an adjacent tooth and (ii) a tooth in vertical contact, the method includes iteratively: adjusting positioning of the tooth in the digital denture model to resolve interproximal (IP) contacts, and adjusting vertical positioning of the tooth.

### INCORPORATION BY REFERENCE

This application claims priority to U.S. Provisional Application Ser. No. 63/348,217, entitled “Auto-Denture Design System for Denture Replacement,” filed on Jun. 2, 2022, the disclosure of which is incorporated by reference in its entirety.

### TECHNICAL FIELD

This document generally describes computer-automated technology for designing dental appliances, such as dentures, for a patient.

### BACKGROUND

A denture is a dental prosthesis that is made to replace missing teeth. Dentures are often supported by the surrounding soft and hard tissue of a patient's oral cavity. For example, a denture may be designed to fit over and be supported by the patient's gum tissue. Dentures may include a denture base region that is formed from an acrylic material and colored to appear similar to gum tissue. Denture teeth formed from acrylic or other materials may be secured to the denture base. The dentures can be designed by a technician, dentist, or other relevant user to fit the particular patient's oral cavity. Once the dentures are designed, they can be fabricated and/or manufactured, then fitted and inserted into the patient's mouth.

### SUMMARY

This disclosure generally describes systems, methods, and computer-automated techniques for generating a digital denture model for denture design and iteratively and automatically adjusting teeth in the model to design dentures for a particular patient. The disclosed technology can implement one or more machine learning models and/or artificial intelligence (AI) algorithms to perform operations including but not limited to selecting a tooth library for designing the particular patient's dentures, generating a digital denture model with the selected tooth library, defining an arch form and occlusal plane using the model, and identifying datums for each tooth in the model. The disclosed technology can perform operations including but not limited to leveling the teeth based on the datums and relative the occlusal plane, snapping the teeth to the arch form, resolving any interproximal (IP) contacts, and adjusting vertical positioning of the teeth in the model. The disclosed technology can iteratively adjust the teeth based on the IP contacts and/or vertical movement, anchor molars in the model, and then return the computer auto-designed digital denture model. The returned digital denture model can be presented in one or more graphical user interfaces (GUIs) to a relevant user, such as a dentist or technician. The user can adjust the digital denture model if desired. If the user adjusts the model, then the disclosed technology can iterate through one or more of the abovementioned operations to appropriately adjust the model. Once the model is adjusted and approved/finalized, the model can be used to fabricate and manufacture the dentures for the particular patient.

Digital models of dentures can be used to create dentures as well as replacement dentures for patients who have lost or damaged their dentures, or who would like an additional set of dentures. Dentures need to be properly fitted to soft tissue in the patient's mouth for comfort and proper function. The physical appearance, including shape, color, and pattern, of the dentures can also be important to the patient. Designing a replacement denture or an original denture from a scan can be time consuming for a dentist or technician. Each denture and replacement denture may require a custom design to ensure patient fit and satisfaction. Conventional methods for producing these custom designs can be time-consuming and imprecise, and the results can be technician-dependent since much of the design process may rely on the technician making selections and design choices based on visual inspection of existing dentures and/or denture scans. The resulting dentures and replacement dentures frequently may require grinding and other types of adjustments, and can be an imperfect fit, thereby impacting the patient experience. Rework and required readjustments to correct the fit of the designed dentures can drive cost and inefficiency for patients, dentists, dental offices, and laboratories.

The disclosed technology provides for automatic detection of landmarks and characteristics in a digital denture model produced from a scan of dentures and automated design of dentures based on the digital model and detected landmarks and characteristics. The automatic detection of landmarks of teeth in a digital denture scan can enable efficient and accurate selection of a tooth library having a close match to size and shape of teeth in the digital denture scan for a particular patient, and placement of the selected teeth from the library in the digital denture scan for use in preparing the denture design for fabrication and manufacturing. The automatic selection can be more efficient than selection by a technician based on visual inspection and can also reduce variability in dentures designed by different technicians. The resulting denture design can be used to fabricate dentures that are a closer match to existing or original dentures in fit, function, and aesthetics, thereby improving the patient experience and acceptance of the replacement dentures.

For example, the disclosed technology can automatically select an occlusal plane, an arch size and shape, and closest matching tooth library based on a CT or cone beam computed tomography (CBCT) scan of an original denture for the patient. After selection of the matching tooth library, software or other computer systems described herein can determine a position of each tooth in a digital denture model using a closest fit algorithm, such as iterative closest point (ICP). The selected tooth library can additionally be down-sampled prior to fitting to the digital denture model to enhance efficient processing of the model.

The teeth can be auto-populated in the digital denture model by the computer system, and can be displayed in a GUI for further manipulation and/or adjustment by a technician. Using heat maps and/or color maps, fit of the teeth in the digital denture model can be efficiently conveyed to the technician to aid in review of the model and further optional adjustments. During adjustments of teeth positioning, the GUI can present the teeth in the display and allow for movement of a tooth or a group of teeth relative to one another while avoiding interference with other teeth through overlap or collision. In some implementations, the technician follows a process flow in the GUI to accept or adjust the teeth in the model using the ICP algorithms, thereby efficiently completing the design process. Accordingly, the technician can accept computer-automated processes to perform iterative operations in adjusting the teeth in the model until a desired dentures design is achieved.

One or more embodiments described herein include a method for performing an auto-setup of a digital denture model, the method including: accessing, by a computer system, a digital denture model for a patient, the digital denture model including upper teeth and lower teeth, defining, by the computer system, an arch form for the lower teeth in the digital denture model, the arch form being aligned with (i) a buccal side of anterior teeth of the lower teeth and (ii) buccal cusps of posterior teeth of the lower teeth, where a same arch form can be used for the upper teeth, defining, by the computer system, an occlusal plane relative to the arch form for the digital denture model, identifying, by the computer system, a group of datums for each tooth of the upper and lower teeth in the digital denture model, leveling, by the computer system, each tooth of the upper and lower teeth in the digital denture model based on the respective group of datums being positioned relative to the occlusal plane, and snapping, by the computer system, each tooth of the upper and lower teeth in the digital denture model to the arch form. Until a threshold level of movement can be achieved between each tooth and at least one of (i) an adjacent tooth and (ii) a tooth in vertical contact, the method can include iteratively: adjusting, by the computer system, positioning of the tooth in the digital denture model to resolve interproximal (IP) contacts, and adjusting, by the computer system, vertical positioning of the tooth in the digital denture model. The method can also include returning, by the computer system, the digital denture model having the adjusted upper and lower teeth.

The embodiments described herein can optionally include one or more of the following features. For example, leveling, by the computer system, each tooth of the upper and lower teeth based on the respective group of datums being positioned relative to the occlusal plane can include: rotating the tooth around a line perpendicular through marginal ridge datums of the tooth. Leveling, by the computer system, each tooth of the upper and lower teeth based on the respective group of datums being positioned relative to the occlusal plane can include: torqueing the tooth using buccal and distal cusp tip datums of the tooth. Leveling, by the computer system, each tooth of the upper and lower teeth based on the respective group of datums being positioned relative to the occlusal plane can include: tipping the tooth mesially or distally using marginal ridge datums of the tooth. As another example, leveling, by the computer system, each tooth of the upper and lower teeth based on the respective group of datums being positioned relative to the occlusal plane can include: identifying a reference plane defined by the group of datums on an anterior tooth, and tipping the anterior tooth according to the reference plane at a pivot point of the anterior tooth. Tipping the anterior tooth can include leveling a tip of the anterior tooth with the occlusal plane.

In some implementations, leveling, by the computer system, each tooth of the upper and lower teeth based on the respective group of datums being positioned relative to the occlusal plane can include: identifying a pivot point for a posterior tooth as a midpoint between 2 marginal ridge datums for the posterior tooth, and rotating the posterior tooth around a line perpendicular to the midpoint to level the posterior tooth with the occlusal plane. The method can also include torqueing the posterior tooth using at least one of buccal cusp datums and distal cusp datums to cause a cusp of the posterior tooth to be parallel to the occlusal plane. The method can also include tipping the posterior tooth in at least one direction of mesially and distally using the 2 marginal ridge datums for the posterior tooth.

As another example, snapping, by the computer system, each tooth of the upper and lower teeth to the arch form can include: for each tooth from a midline to a last molar in the upper teeth, snapping the tooth tangent to the arch form, and for each tooth from the midline to a last molar in the lower teeth, snapping the tooth tangent to the arch form. For each tooth from a midline to a last molar in the upper teeth, snapping the tooth tangent to the arch form can include: snapping central incisors to the midline and tangent to the arch form, snapping lateral teeth, snapping canines so that respective cusp tips of the canines can be positioned (i) relative to a tangent line on the arch form or (ii) a threshold distance outside of the arch form, and for each molar and upper bicuspid tooth, (iii) rotating the tooth so that respective marginal ridge datums can be tangent to the arch form and (iv) positioning the tooth buccal-lingually so that the marginal ridge datums can be aligned on the arch form. In some implementations, for each tooth from the midline to a last molar in the lower teeth, snapping the tooth tangent to the arch form can include: snapping lower incisors to the midline and inside a tangent line so that the arch form can touch a buccal side of the lower incisors, snapping lower canines so that cusp tips of the lower canines can be positioned (i) relative to the tangent line inside the arch form or (ii) a threshold distance inside of the arch form, and for each molar and lower posterior tooth, translating the tooth lingually so that a respective buccal cusp tip can be positioned on the arch form.

In some implementations, adjusting, by the computer system, positioning of the tooth to resolve interproximal (IP) contacts can include: for each tooth, generating a bounding box, for each tooth, identifying a center point of the tooth as a center point in the bounding box, identifying a vector between center points of the teeth, selecting the tooth at a defined position, the defined position being a midline, and moving the tooth along the vector between the tooth and the adjacent tooth to (i) maintain relative orientation, remove overlap, and (ii) put the tooth in contact with the adjacent tooth at a predefined contact point.

Sometimes, the method can also include iteratively adjusting the vector between each next set of adjacent teeth and iteratively moving each next set of adjacent teeth along the respective vector until a last tooth is moved. The method can also include selecting a second tooth at a second defined position, the second defined position being a side of the midline that is opposite the defined position of the tooth, and iteratively moving teeth adjacent the second tooth until a last tooth on the side of the midline that is opposite the defined position of the tooth is moved.

Adjusting, by the computer system, vertical positioning of the tooth can include moving each tooth of the lower teeth in a direction perpendicular to the occlusal plane until the tooth contacts the occlusal plane. Adjusting, by the computer system, vertical positioning of the tooth can include socking each tooth of the upper teeth until the tooth contacts one or more of the lower teeth. Sometimes, adjusting, by the computer system, vertical positioning of the tooth can include: identifying a center point between 3 adjacent teeth to define a buccal vector as perpendicular to the center point, for each tooth, adjusting the tooth buccally, lingually, and down based on the buccal vector, measuring a distance between the adjusted tooth and at least one tooth vertically in contact with the adjusted tooth, determining whether the distance is within a predetermined threshold distance, reducing the distance in half based on determining that the distance is not within the predetermined threshold distance, moving the adjusted tooth in an opposite direction of the adjustments by the reduced distance, measuring a new distance between the adjusted tooth and the at least one tooth vertically in contact with the adjusted tooth, and iteratively moving the adjusted tooth buccally, lingually, down, and up until the measured distance is within the predetermined threshold distance.

In some implementations, the method can also include receiving, by the computer system, patient tooth data that can include at least one image of teeth of the patient, selecting, by the computer system and from a data store, a candidate tooth library from amongst a group of static tooth libraries based at least in part on the patient tooth data, and generating, by the computer system, the digital denture model based on the patient tooth data and the candidate tooth library. Generating the digital denture model can include overlaying teeth of the candidate tooth library over corresponding teeth of the digital denture model. The method can also include transmitting the digital denture model to a user device for presentation in a graphical user interface (GUI) at the user device, receiving, by the computer system and from the user device, user input indicating one or more adjustments to at least one tooth of the upper teeth and the lower teeth in the digital denture model, and iteratively performing, by the computer system and based on the user input, at least one of: (i) leveling the at least one tooth, (ii) snapping the at least one tooth to the arch form, and (iii) until a threshold level of movement can be achieved between the at least one tooth and at least one of (a) an adjacent tooth and (b) a tooth in vertical contact, adjusting a position of the at least one tooth to resolve IP contacts and adjusting a vertical positioning of the at least one tooth. Sometimes, snapping, by the computer system, each tooth of the upper and lower teeth to the arch form can include aligning the tooth to the arch form using an iterative fitting algorithm, the iterative fitting algorithm being an iterative closest point algorithm.

For example, in one implementation, a method of designing a digital denture model includes receiving a scan of an existing denture, converting the scan of the existing denture to a digital denture model, selecting a tooth mold from a plurality of tooth libraries based on one or more landmarks of teeth of the digital denture model, identifying anatomic landmarks of the teeth of the digital denture model, selecting a tooth from the tooth mold and positioning the selected tooth in the digital denture model overlaying a corresponding tooth of the digital denture model based on the identified anatomic landmarks, and aligning the tooth to a identified arch and occlusal plane of the digital denture model. The automation of parts of the design process can improve the efficiency of designing and producing replacement dentures that replicate the fit, function and appearance of existing dentures.

In another implementation, a method of designing a digital denture model includes confirming, in a user interface, an automatically selected tooth mold for use with a digital denture model prepared from a scan of an existing denture, adjusting, in the user interface, an alignment of a tooth of the selected tooth mold in the digital denture model by selecting the tooth and moving the tooth within the digital denture model using a user input device, wherein a movement of the tooth within the digital denture model is constrained by positions of other teeth of the model where the movement of the tooth does not interfere with or overlap with the other teeth of the model, and confirming, in the user interface, an adjusted alignment of the tooth, wherein the confirmation indicates an approval of the alignment of the tooth to an occlusal interface and to an arch of the digital denture model.

Certain implementations may provide one or more advantages. In a first example, the disclosed technology can generate improved and better denture designs over technician-based designs, and can do so in a manner that is computationally efficient (e.g., uses minimal computational resources, such as CPU cycles, RAM, network bandwidth). For example, a challenge that is posed with traditional denture design is a multitude of potential variation in orientation and positioning of teeth in 3D space relative to each other and to opposing archways—resulting in a near infinite number of possible arrangements and setups for consideration. The disclosed technology can be used to iteratively prune a universe of possible teeth arrangements to efficiently arrive at an appropriate denture design for a patient in a manner that minimizes the computational resources required to perform such techniques.

In a second example, using software to automate portions of the denture design process improves efficiency over technician development of denture designs based on visual identification of denture landmarks. A CT scanning device, CBCT scanning device, or other scanning device can quickly produce a scan of existing dentures that can be used by a computer program to identify a matching tooth library, automatically determine various denture landmarks in a digital model produced from the scan, and position and align teeth from the tooth library in the digital model. The process for designing dentures can be significantly longer if a technician completes these steps based on visual inspection of the existing dentures or existing denture scan and manual manipulation of a denture model.

In a third example, automating the denture design process can enable standardization of the process of producing a digital denture design. Landmarks and best-fits can be efficiently determined using the disclosed technology and iterative best fit algorithms can be applied to improve accuracy and efficiency in designing the dentures. The automatically produced denture designs can be of higher-quality than technician-produced designs, especially since the auto-designed dentures can more closely replicate the design of existing dentures and/or the patient's teeth in scan data. The disclosed technology can also reduce variability in denture designs made by different technicians who generate designs by visual and manual inspection. In turn, automating the design process can result in denture designs that are accurate and thus used to manufacture dentures having desired fit, function, and appearance characteristics, thereby increasing patient acceptance and approval of the dentures.

In a fourth example, automating the design process as described herein can significantly speed up the design process by suggesting libraries of teeth that are a close match to the teeth of the existing dentures, and automatically adjusting positions and orientations of teeth in the denture design to mimic existing dentures or a desired fit and appearance of the dentures for the patient. Because the program can make suggestions and automatic adjustments of best-fit teeth positions and selections, the work that is required of a technician can be significantly reduced. The technician can simply review the suggestions and make minor adjustments, if needed, to the digital model rather than create an entire model from scratch. The amount of technician time required for creating a digital denture design can be decreased and throughput of denture designs can be increased. Designs for dentures can be sent for manufacturing more quickly, and patients can receive their dentures quickly and at less cost due to the automated process reducing time required in the overall design process. Similarly, automating the techniques described herein allows for less-skilled technicians or other users to review and design dental appliances.

Moreover, the digital denture teeth can remain in contact as they are automatically positioned, thereby increasing efficiency for both a user and a computer system in positioning the digital denture teeth. For example, all posterior teeth can be adjusted into occlusion together, with each tooth moving so as to maintain occlusion as the teeth are moved along the arch of the denture. The teeth maintain their relationship to one another during the movement into occlusion in one dimension while they move and pivot in other dimensions to maintain contact with the opposing surface. Fewer processing cycles may be used to automatically move a tooth into contact than would be used to generate a user interface and receive user inputs to position the digital denture tooth in contact.

Another benefit of automatically moving the digital denture tooth into contact is that the resulting arrangement of digital denture teeth may be more consistently of high quality than an arrangement where each digital denture tooth is moved into contact by a user. In some implementations, multiple digital denture teeth may be selected and moved together to improve computing efficiency and accuracy in moving and aligning teeth to achieve a preferred denture design and model. Traditionally, a dentist may spend significant amounts of time visually analyzing patient tooth data with color or other visual maps to determine if neighboring and/or opposing teeth are touching or otherwise in preferred contacts. The disclosed technology, on the other hand, provides for automatically checking and resolving teeth contacts so that the teeth are appropriately aligned/in contact. These automated techniques can speed up the design process while maintaining accuracy and high quality arrangement of teeth for any patient.

Additional advantages will be apparent to the person of skill in the art based on the figures, description, and claims herein.

### DESCRIPTION OF THE DRAWINGS

FIG. 1 is a conceptual diagram of a system for computer-automated design of dentures for a patient.

FIG. 2 is a flowchart of an example process for automatic design of a replacement denture.

FIGS. 3A, 3B, and 3C are a flowchart of a process for computer-automated design of dentures for a patient.

FIG. 4 is an example user interface screen (GUI) that may be generated by digital denture design system with labels on identified landmarks (e.g., datums) of teeth in a digital denture model.

FIG. 5A is an example GUI that may be generated by a digital denture design system for positioning of an occlusal plane relative to a digital denture model.

FIG. 5B is an example GUI that illustrates adjustments made to the occlusal plane relative to a digital denture model.

FIG. 5C illustrates example teeth in a digital denture model before being adjusted relative to an occlusal plane for the model.

FIG. 5D illustrates the example teeth in the digital denture model of FIG. 5C after being adjusted relative to the occlusal plane for the model.

FIG. 6A illustrates example adjustments of rotating, tipping, and/or torqueing one or more teeth in a digital denture model.

FIG. 6B illustrates example tipping adjustments that can be made to teeth in a digital denture model.

FIG. 6C illustrates example torqueing adjustments that can be made to teeth in a digital denture model.

FIG. 7A is an example GUI that may be generated by the digital denture design system for identifying an arch of a digital denture model.

FIG. 7B is an example GUI that may be generated by the digital denture design system for providing options to a technician to aid in adjusting an arch of a digital denture model.

FIG. 7C illustrates snapping one or more lower teeth to an arch of a digital denture model.

FIG. 7D illustrates an arch form for both upper and lower teeth of a digital denture model.

FIG. 8 illustrates adjusting teeth in a digital denture model to resolve IP contacts.

FIGS. 9A and 9B illustrate example movements for socking, vertically positioning, and/or correcting overbite in a digital denture model.

FIG. 10 is a flowchart of a process for automatically leveling teeth in a digital denture model.

FIG. 11 is a flowchart of a process for automatically snapping teeth to an arch form of a digital denture model.

FIG. 12 is a flowchart of a process for automatically resolving IP contacts in a digital denture model.

FIG. 13 is a flowchart of a process for automatically socking upper teeth in a digital denture model.

FIGS. 14A and 14B illustrate example GUIs that may be generated during an automated process of positioning selected tooth library teeth in a digital denture model and automatically aligning and leveling each tooth of the selected tooth library.

FIGS. 15A, 15B, and 15C illustrate example GUIs that may be generated during a process of automatically aligning and leveling each tooth of a selected tooth library in a digital denture model.

FIG. 16 illustrates an example GUI that may be generated during a process of manually adjusting a position of a selected tooth of a tooth library in a digital denture model.

FIG. 17 illustrates an example GUI that may be generated during a process of manually adjusting a position of a selected group of teeth of a tooth library in a digital denture model.

FIGS. 18A and 18B illustrate example GUIs including toolboxes with user-selectable options that may be used while designing and/or adjusting a digital denture model.

FIG. 18C illustrates example GUIs with respective toolboxes of selectable options for automatically designing and/or adjusting lower and upper teeth, respectively, in a digital denture model.

FIGS. 19A, 19B, and 19C illustrate schematic diagrams of an example digital denture model and example denture teeth.

FIG. 20A illustrates example libraries of digital denture teeth.

FIG. 20B illustrates differences between the digital denture teeth of FIG. 20A with an overlay color map.

FIG. 21 is a flowchart of an example process for automatic design and fabrication of a replacement denture.

FIG. 22 is a conceptual diagram of system components for selecting tooth libraries.

FIG. 23 is an example architecture of a computing device, which can be used to implement aspects according to the present disclosure.

Like reference symbols in various drawings indicate like elements.

### DETAILED DESCRIPTION

The disclosed technology generally provides systems, methods, and techniques for automatically designing dentures and replacement dentures for a patient based on digital models developed from scans of existing dentures and/or the patients mouth and with iterative fitting algorithms. The use of iterative fitting algorithms can improve accuracy and efficiency of a process for designing the dentures. The speed with which a computer system can identify one or more matching tooth libraries from a scan of digital dentures can be faster than a conventional approach of visual identification of a library by a technician. The computer system can identify multiple landmarks of the digital model to determine the appropriate library, which can provide for auto-designing dentures that are more likely to achieve fit, function, and appearance criterion for the patient. Technicians can make adjustments to the digital model more efficiently using the disclosed technology, which can allow for manipulation of groups of teeth together while also preventing interference between the teeth and/or other discrepancies in the dentures design. The use of the technologies described herein can provide a more efficient process for producing digital denture designs for dentures.

Replacement dentures may be needed if original dentures, and/or an acrylic base of the dentures, are damaged, stained, and/or lost over time. A patient or doctor may desire that an arrangement of teeth in the replacement denture be similar to the original denture. It may be difficult and time consuming, however, to provide similar denture teeth and a similar arrangement of denture teeth using conventional techniques for fabricating replacement dentures. For example, neither the patient nor dentist may know which type of denture teeth (e.g., which library of denture teeth) were used in fabricating the original dentures (e.g., due to a patient changing dentists or records being lost). Additionally, traditional techniques for replicating a shape of the base of the original denture may be time consuming, imprecise, and/or use significant amounts of consumable materials to, for example, build molds of the original denture that can then be used to form a new, similar denture base.

Moreover, because conventionally a technician designs each replacement denture based on visual inspection, there may be a backlog of dentures to design, resulting in long wait times for replacements. A shortage of technicians trained to design the dentures adds to the length of time between a patient needing a replacement dentures and receiving the dentures, which can lead to prolonged patient discomfort or additional dental issues. The reliance on technicians to design replacement dentures increases labor costs associated with the creation of dentures and contributes to the high cost of quality dentures.

Different technicians may make design selections differently or based on different criteria, resulting in variation in dentures designed by different technicians. It can be difficult to distinguish dimensional differences between teeth of varying shapes based on visual inspection, and requiring a technician to make these determinations can create numerous challenges. The guesswork sometimes required of a technician in determining a library of teeth that match a denture scan can cause differences in fit and function between original and replacement dentures. The technician selection of library teeth based on visual inspection of the denture scan may also introduce variability in the output design and manufactured dentures. The dentures can look different when the selected teeth vary from the teeth design of the original dentures, and can fail to meet the expectations of patients and their loved ones. The disclosed technology provides techniques to auto-design dentures using digital denture models and iterative algorithms so that dentures can be efficiently and accurately designed as well as manufactured according to a patient's requirements for the dentures.

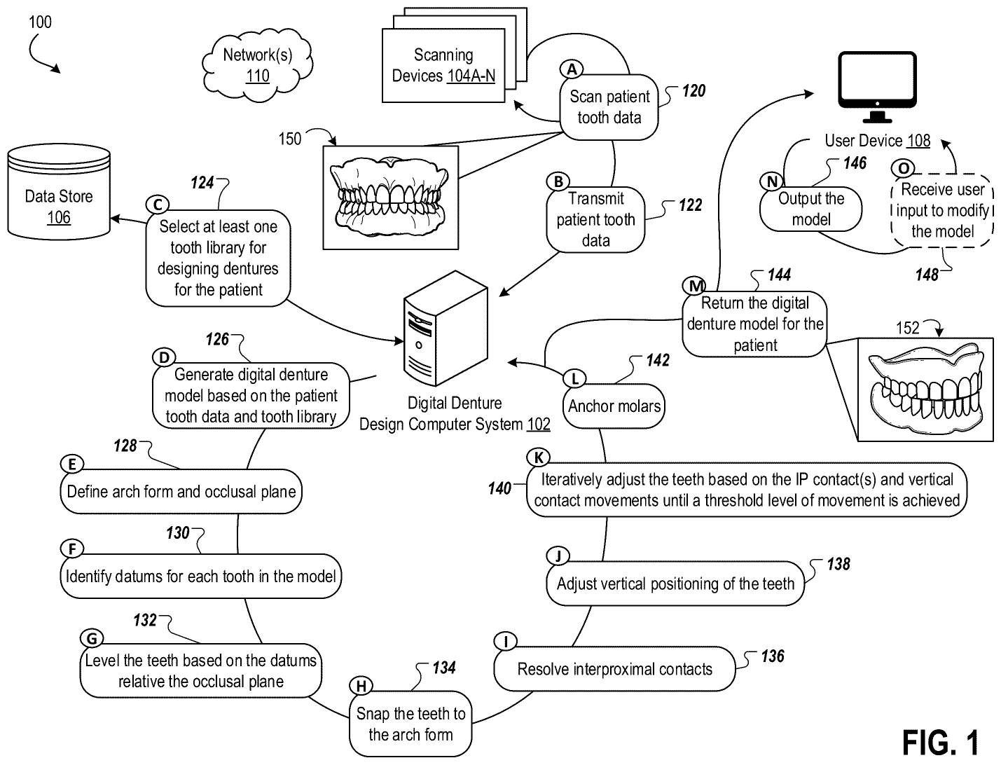

Referring now to the figures, FIG. 1 is a conceptual diagram of a system 100 for computer-automated design of dentures for a patient. The system 100 can include a digital denture design computer system 102 (e.g., the ‘computer system’), scanning devices 104A-N, a data store 106, and/or a user device 108 that communicate (e.g., wired, wirelessly) with each other via network(s) 110.

The computer system 102 can be any type of computing system, cloud-based computing system, computing device, and/or network of computer systems. The computer system 102 can be remote from dental offices or other locations where a patient may go to get fitted for and receive dental appliances, such as dentures. In some implementations, the computer system 102 can be in the dental office and/or part of the user device 108 or other computer system in the dental office. The computer system 102 can be configured to generate dental appliance designs unique to each patient based on processing patient-specific data, selecting tooth libraries that satisfy one or more selection criteria, generating a digital denture model for the patient, and using the selected tooth libraries to design dental appliances for the patient in the model with iterative algorithms.

The scanning devices 104A-N can include but are not limited to intraoral scanners, computed tomography (“CT”) scanning devices, cone beam computed tomography (“CBCT”) scanning devices, and/or other types of 3D imaging devices that may be used to capture images and other scan data of patients' mouths, dentition, jaws, and/or faces, such as patient scan data 150. The scan data 150 can be a 3D image or other type of representation of the patient's mouth and/or existing dentures. In some implementations, the scan data 150 can be previously captured using the scanning device(s) 104A-N (e.g., when the patient visits their dentist's office), then stored in the data store 106.

The data store 106 can be any type of database, data store, data repository, and/or cloud-based storage system. The data store 106 can store many different tooth libraries that have been predefined and/or previously generated, which can be used by the computer system 102 to auto-design dentures for patients. The tooth libraries can be generic and applicable to all patients. In some implementations, the tooth libraries can be generated for particular types of teeth, particular groups of teeth, particular purposes (e.g., dental implants, dental inserts, caps, crowns, veneers, dentures), particular patient demographics (e.g., age, gender, dental condition), etc. The tooth libraries can each contain data or metadata, which further can be used with the disclosed techniques. For example, each tooth library can include a predefined coordinate system. The coordinate system can then be used by the computer system 102 in determining measurements of the respective tooth and/or arranging and setting up the tooth relative to other teeth for the patient's digital denture design model. Each tooth library can include additional or other information, including but not limited to tooth measurements, color data, shape data, texture data, etc. The data store 106 may also store patient data, including but not limited to patient information (e.g., age, gender, teeth condition, dental appliance type, procedure, and/or history, and/or demographic information), 3D image data (e.g., captured by the scanning devices 104A-N, such as the scan data 150), 2D image data (e.g., converted from the 3D image data by the computer system 102), teeth scans (e.g., as generated or captured by the scanning device(s) 104A-N or other teeth imaging devices), and/or teeth measurements (e.g., as provided by a relevant user and/or automatically determined by the computer system 102). Various other information described herein can also be stored in the data store 106, such as previously made digital denture models and/or historic information about previous dentures of the patients or other dental appliances, all of which can be used by the computer system 102 to auto-design new and/or replacement dentures or other dental appliances for the patients.

The user device 108 can be any type of computing device including but not limited to smartphones, tablets, laptops, computers, mobile phones, mobile devices, and/or wearable devices. The user device 108 can be used by a relevant user, such as a technician, dentist, and/or orthodontist. The user device 108 can be configured to present and output information in GUIs about auto design of the dentures for the patient to the relevant user. The user device 108 can also be configured to receive user input indicating selection of tooth libraries and/or manual manipulation of the design of the dentures, as described further herein.

Still referring to the system 100 in FIG. 1, the scanning devices 104A-N can scan patient tooth data, such as the scan data 150 (block A, 120). The scanning can be performed at any time before the computer system 102 auto-designs the dentures for the patient. The scanning devices 104A-N can then transmit the patient tooth data to the computer system 102 in block B (122). In some implementations, the patient tooth data can be stored in the data store 106 and then the computer system 102 can retrieve the patient tooth data at a time at which the computer system 102 auto-designs dentures for a particular patient. In some implementations, the scan data 150 can be transmitted first to the user device 108 and reviewed by the technician before the user device 108 transmits the patient tooth data to the computer system 102 for further processing. Sometimes, the computer system 102 can request the scan data 150 or other patient tooth data once the technician provides user input at the user device 108 indicating a desire to design dentures for a particular patient. Other times, the computer system 102 can automatically receive the patient tooth data when the patient tooth data is generated (e.g., when scans or images are taken of the patient's teeth and/or mouth) and/or at predetermined times (e.g., every 15 minutes, every 30 minutes, every 1 hour, every hours, every 12 hours, every 24 hours).

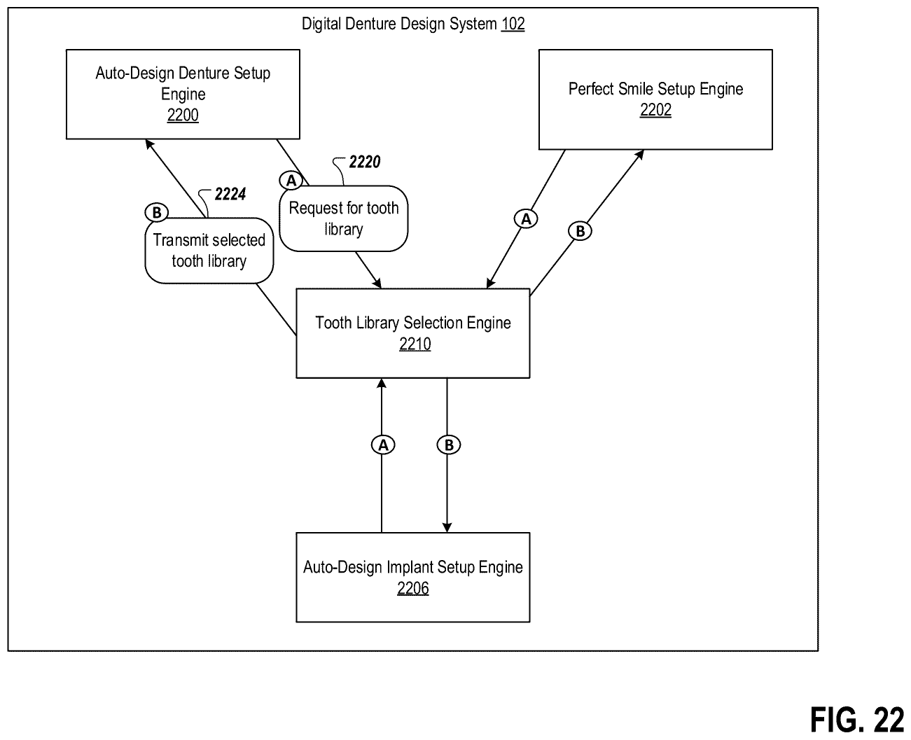

The computer system 102 can select at least one tooth library for designing the dentures for the patient in block C (124). The computer system 102 can perform techniques such as measuring teeth in the patient tooth data and using those measurements to identify tooth libraries in the data store 106 having similar tooth measurements (or measurements within a threshold range of the measurements for the particular patient). The computer system 102 can select a candidate tooth library amongst a set of tooth libraries based on identifying a best fitting tooth library for the particular patient using machine learning models and/or artificial intelligence (AI) algorithms. The computer system 102 can score each of the set of tooth libraries and select a highest-scoring tooth library as the candidate tooth library. The computer system 102 can additionally or alternatively consider tooth shape, tooth lengths, tooth widths, and/or aggregate tooth measurements in selecting the candidate tooth library. Refer to at least FIG. 22 for further discussion.

Once the computer system 102 selects the at least one tooth library, the computer system can generate a digital denture model for the patient based on the patient tooth data and the at least one tooth library (block D, 126). The computer system 102 can load the selected tooth library into the digital denture model. The loaded teeth may not be in any designated positions in the model, nor may the teeth be aligned according to one or more arch forms, occlusal planes, or relative to each other, in some implementations. In some implementations, generating the digital denture model can include overlaying teeth of the selected/candidate tooth library over corresponding teeth of the digital denture model.

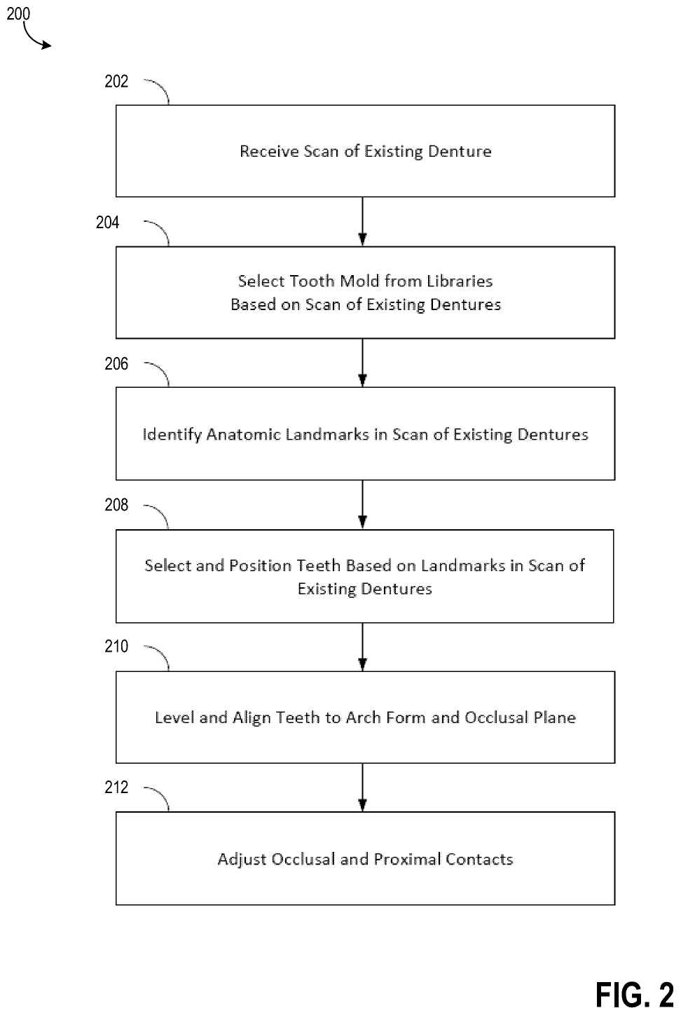

Refer to at least FIGS. 2, 14A, 14B, 15A, 15B, 15C, 19A, 19B, 19C, 20A, 20B, and 21 for further discussion. In some implementations, the computer system 102 can first generate the digital denture model based on the patient tooth data (block D, 126) and then select the at least one tooth library for designing the patient's dentures with the model (block C, 124).

Using the digital denture model, the computer system 102 can define an arch form and/or occlusal plane in block E (128). The arch form can be automatically generated by the computer system 102. In some implementations, as described herein, the relevant user, such as a technician, may also adjust the arch form on the digital denture model. For example, a curved line can be visually displayed over lower (or upper) teeth of the model. One or more selectable points/spheres (e.g., nodes) can also be presented as part of the curved line over the lower teeth of the model. The user can select, click, and/or drag any of the selectable points to adjust a shape of the curved line. The shape of the curved line corresponds to the defined arch form. The user can, for example, drag one or more of the selectable points along the curved line to set a portion of the curved line to go through a buccal side of anterior teeth in the model and to go through buccal cusps of posterior teeth in the model. When the user adjusts one point on one side of the curved line, the computer system 102 can automatically adjust another point opposite the user-adjusted point on an opposite side of the curved line. Therefore, the teeth of the model can be adjusted to be symmetrical. In some implementations, if desired, the user can select an option for an asymmetrical curve and thus adjustments on the one side of the curved line may not be mirrored on the opposite side of the curved line.

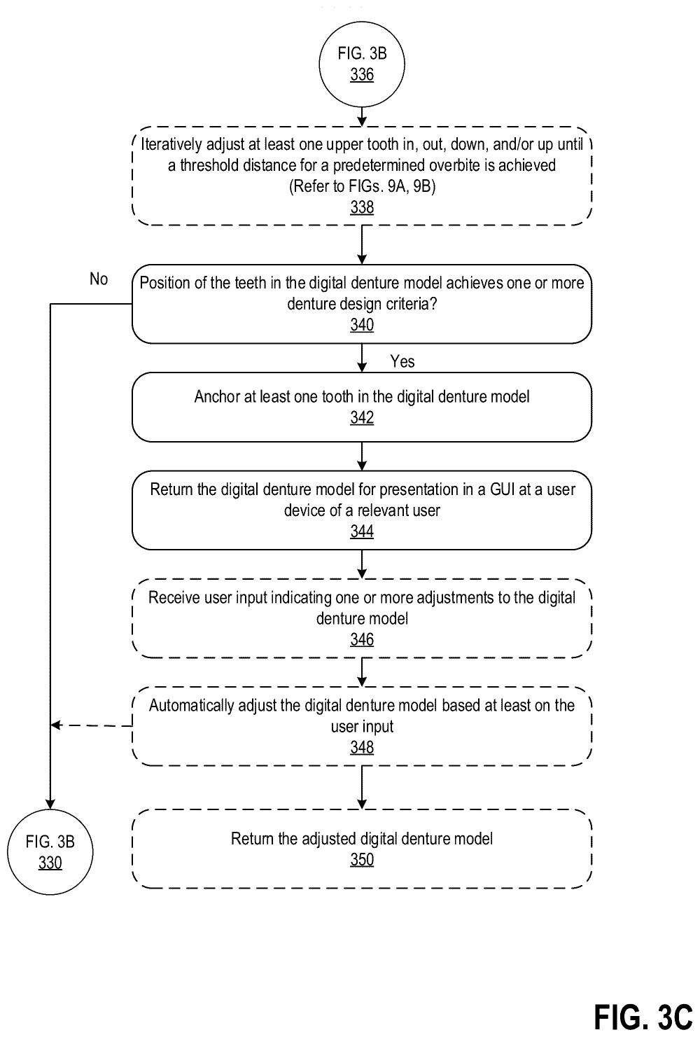

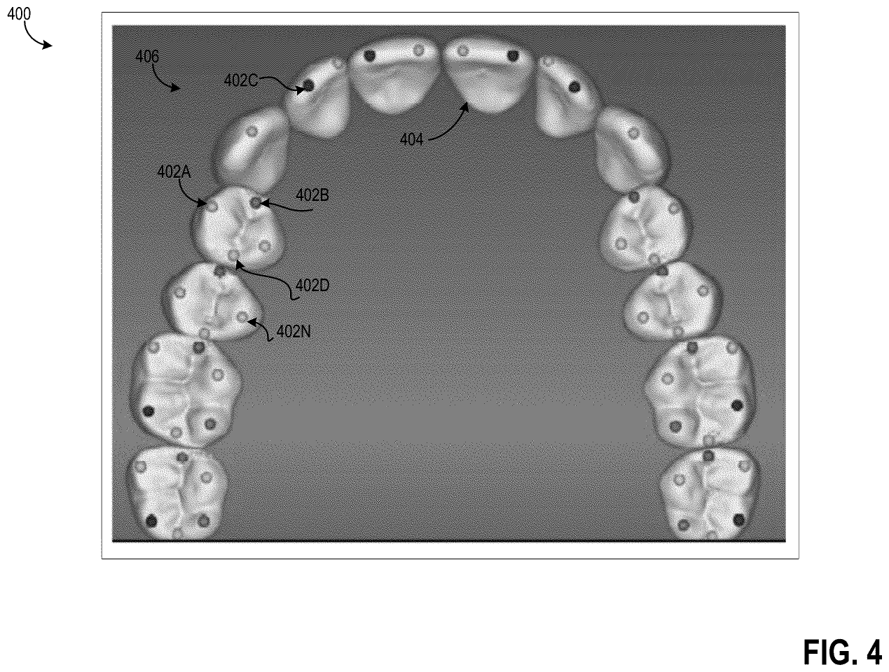

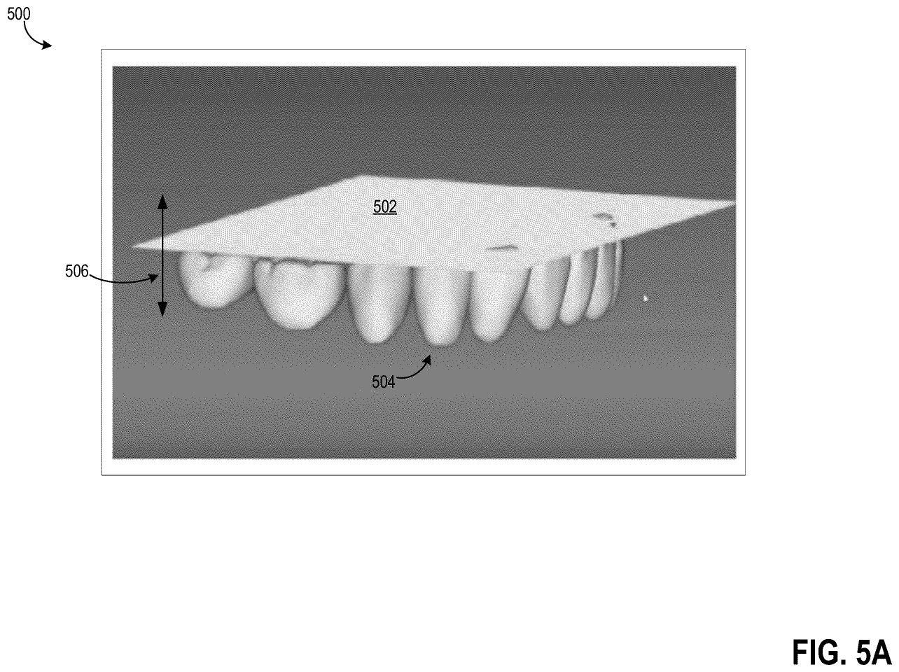

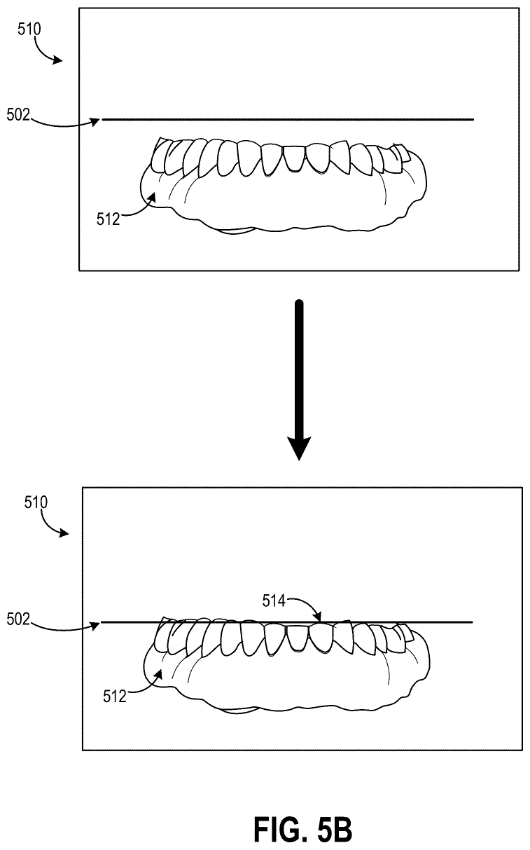

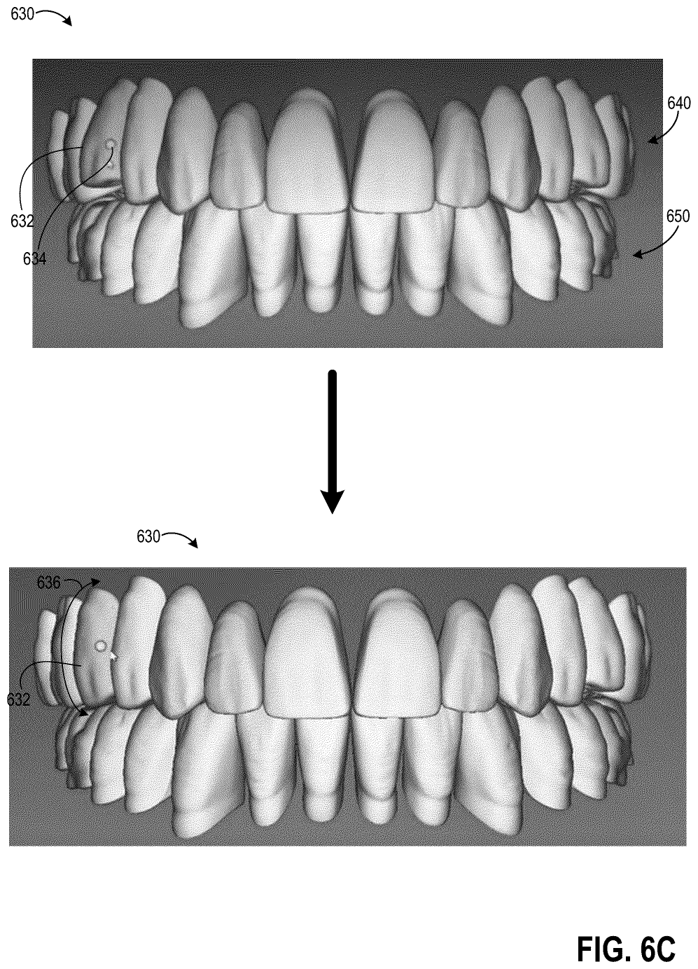

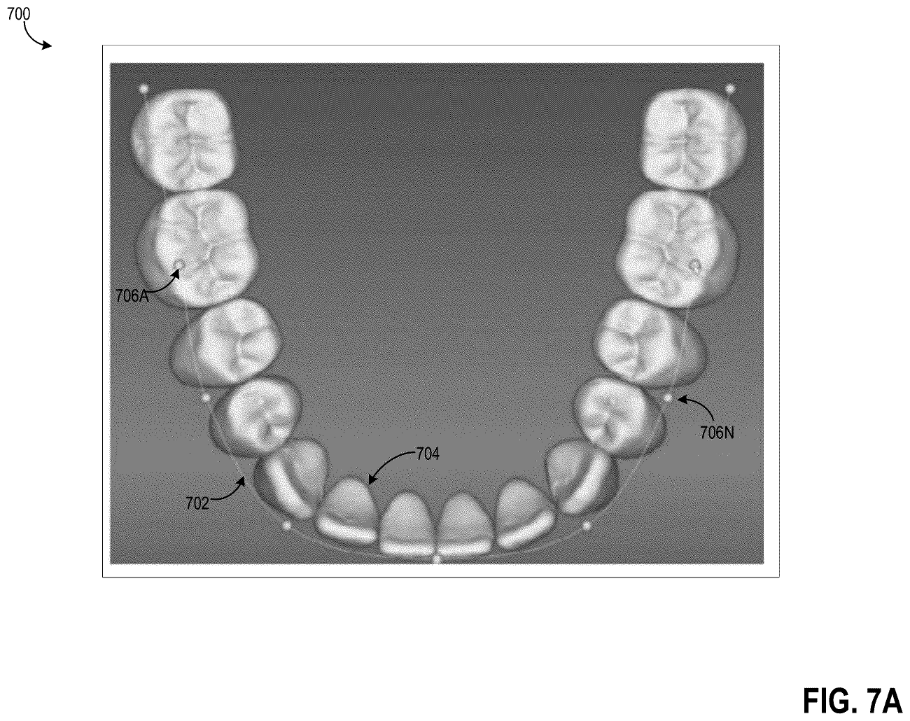

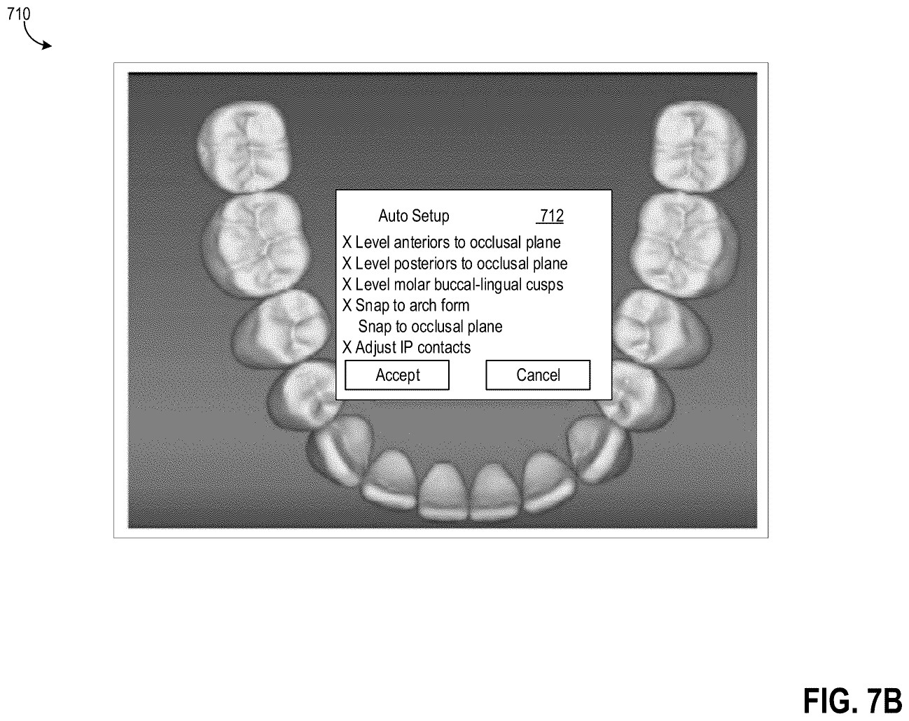

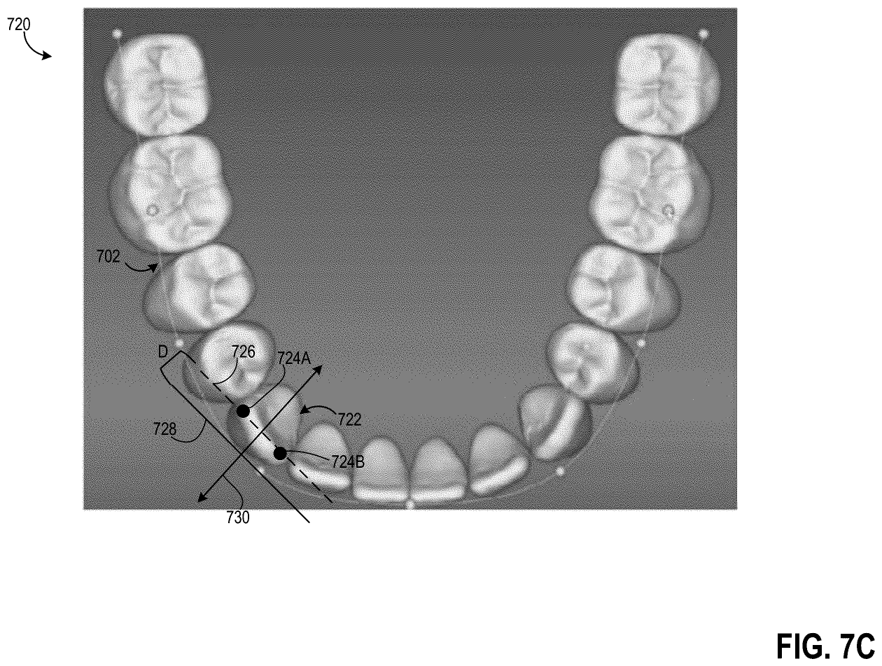

The computer system 102 can define a lower arch form first. The lower arch form can be mirrored for an upper arch form for inner arch coordination. Once mirrored, the user can optionally adjust the upper arch form. For example, the user or the computer system 102 can move the upper arch form to be positioned over an inside of a midline of upper teeth. The user and/or the computer system 102 can also adjust the upper arch form such that the corresponding curved line goes down through center grooves of posterior teeth in the upper arch. In some implementations, the lower and upper arch forms can be defined separately and/or differently. In some implementations, the upper arch form can be defined first, then used to define the lower arch form. Refer to at least FIGS. 5A, 5B, 5C, 5D, 7A, 7B, 7C, and 7D for further discussion.

In block E (128), the computer system 102 can also define the occlusal plane. For example, the occlusal plane can be set for the lower teeth of the model. The user can view the lower teeth of the model with the occlusal plane in an anterior view at the user device 108. The user can then select the occlusal plane and optionally move the occlusal plane up and/or down (e.g., by scrolling with a mouse wheel) to a desired position. The user can optionally adjust a cant and/or tip of the occlusal plane as well if the user does not desire the plane to be perfectly horizontal. In some implementations, the computer system 102 can automatically move the occlusal plane as described above.

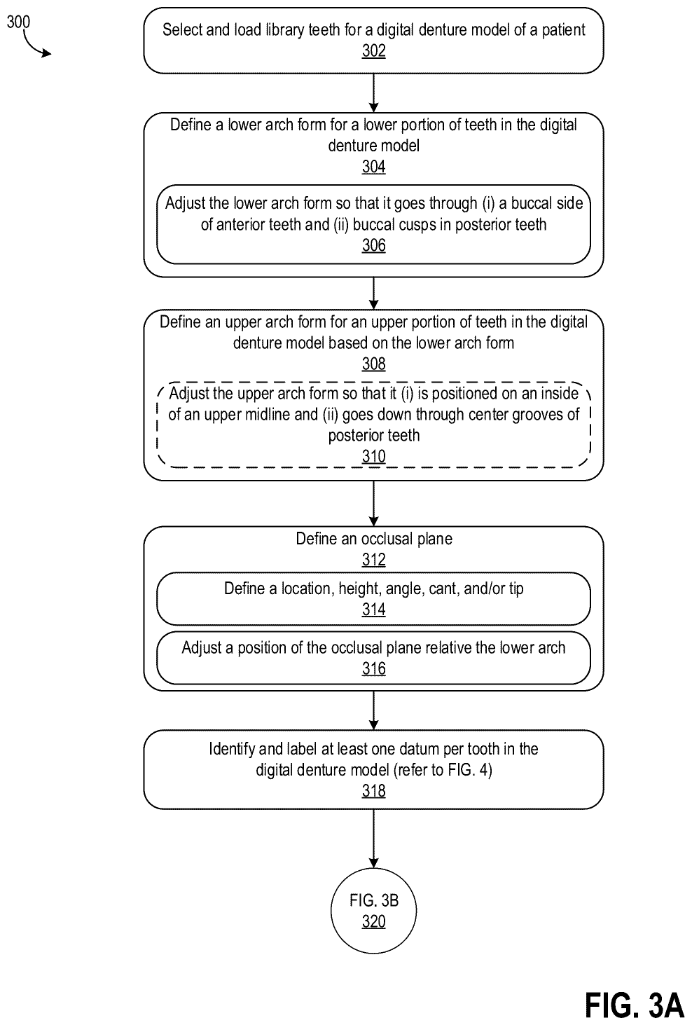

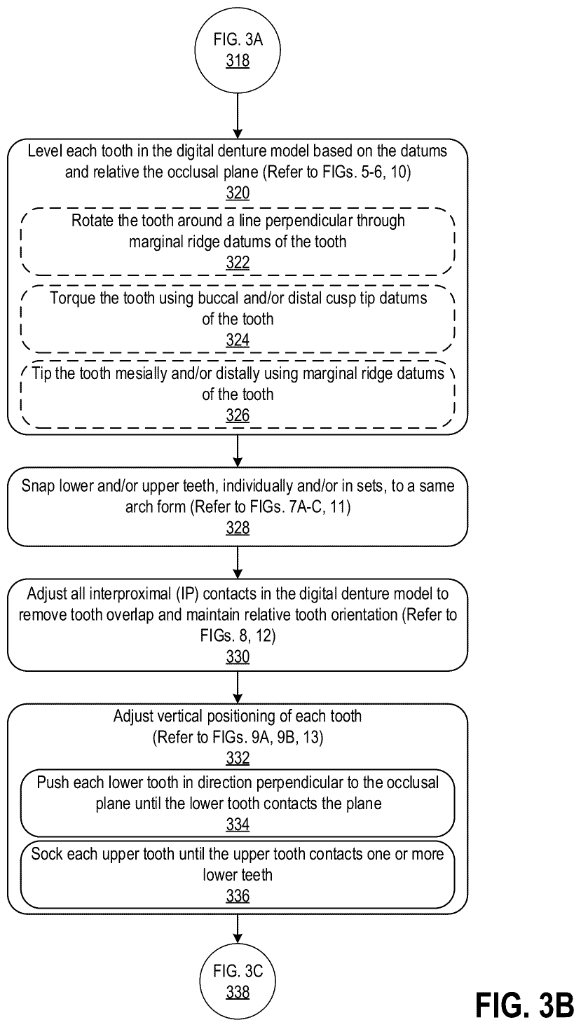

In block F, the computer system 102 can identify a plurality of datums (e.g., landmarks) for each tooth in the model (130). Refer to at least FIGS. 3A, 3B, 3C, and 4 for further discussion.

The computer system 102 can level the teeth in upper and/or lower arches of the model based on the identified datums and/or relative the defined occlusal plane (block G, 132). Refer to at least FIGS. 3A, 3B, 3C, 5A, 5B, 5C, 5D, and 10 for further discussion.

The computer system 102 can also snap the teeth in the upper and/or lower arches of the model to the defined arch form in block H (134). Refer to at least FIGS. 3A, 3B, 3C, 7A, 7B, 7C, and 11 for further discussion.

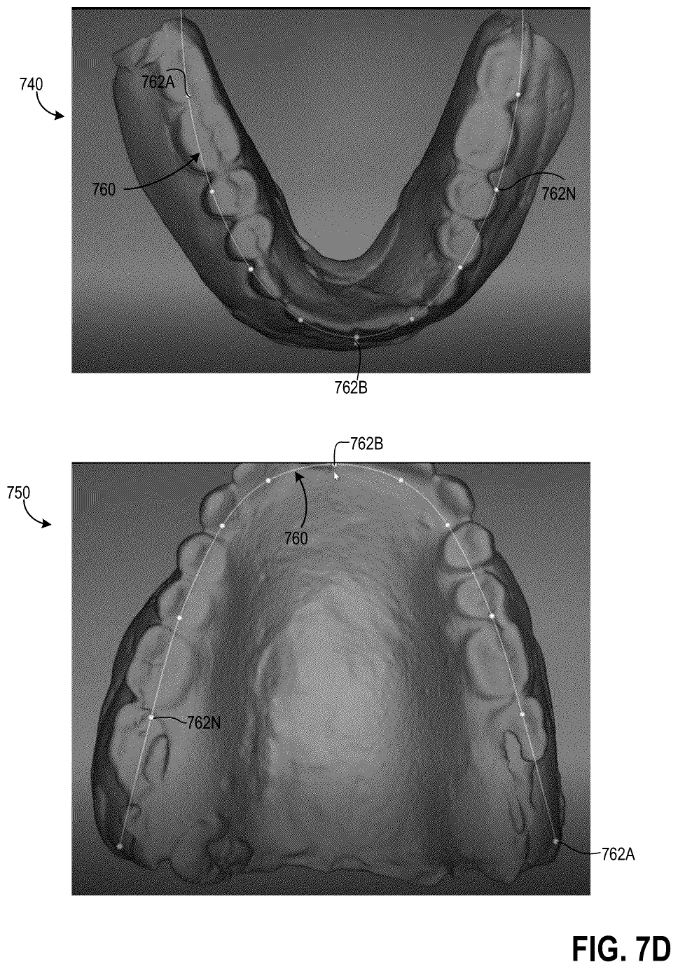

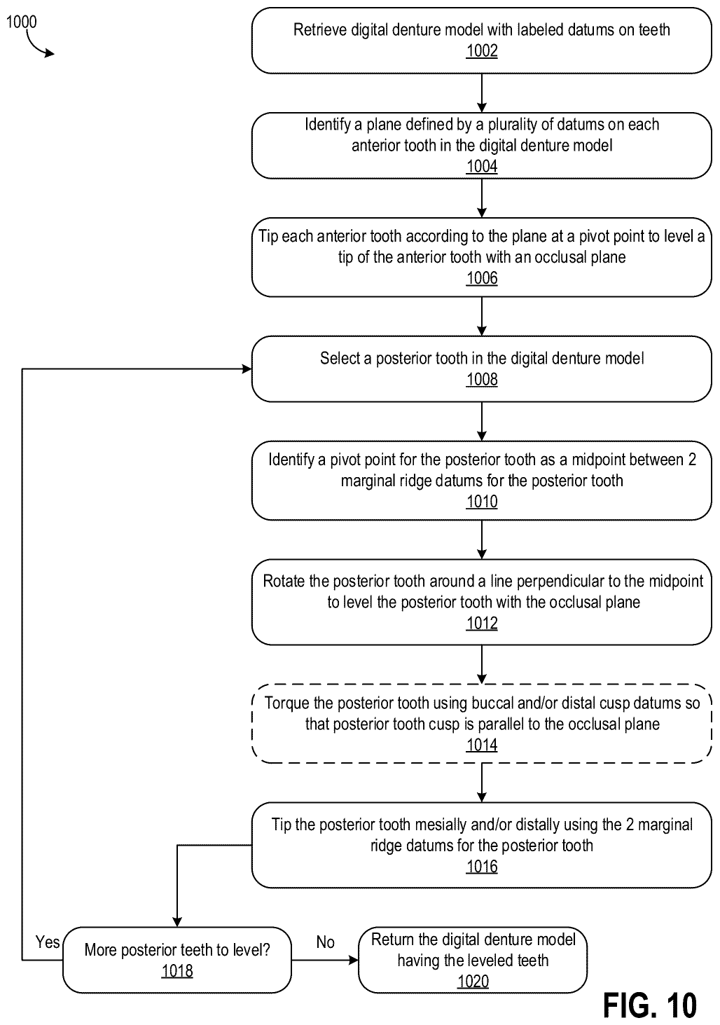

In block I, the computer system 102 can resolve any interproximal (IP) contacts in the model (136). Refer to at least FIGS. 8 and 12 for further discussion.

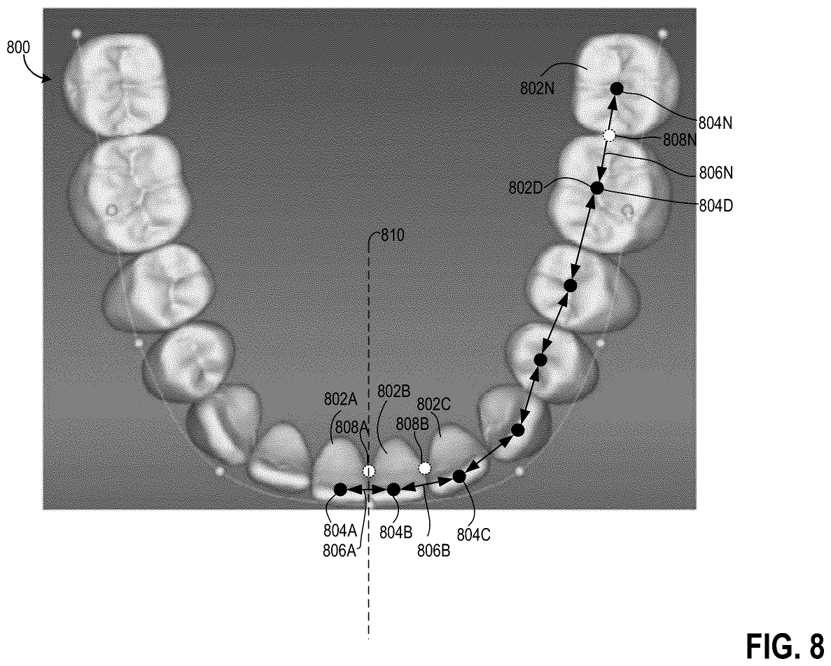

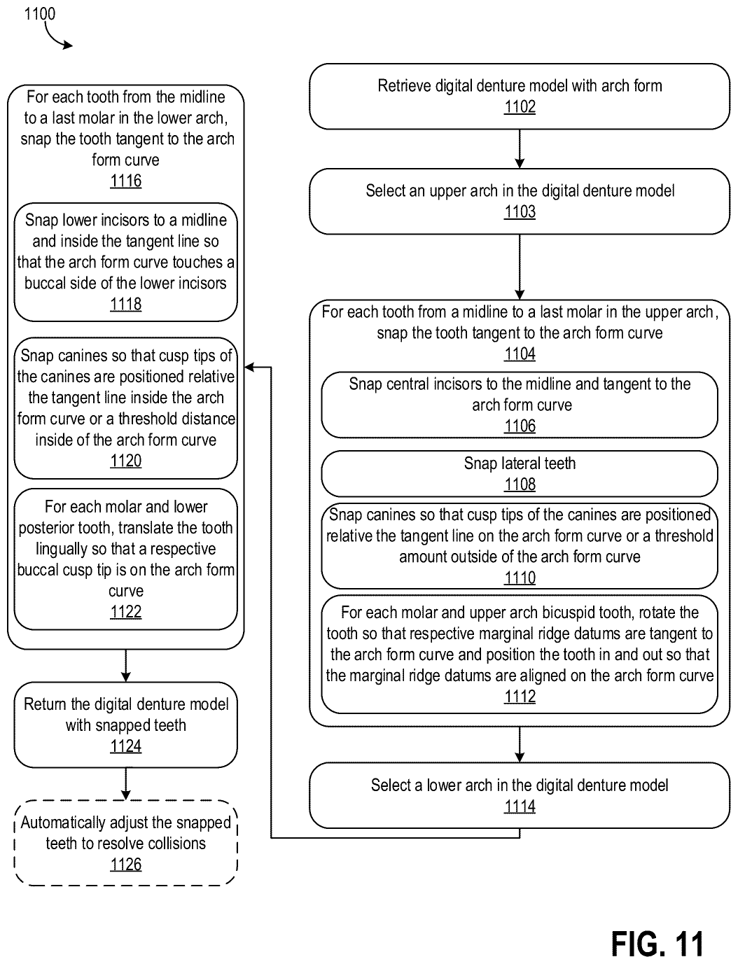

The computer system 102 can also adjust vertical positioning of one or more teeth in the upper and/or lower arches of the model in block J (138). Refer to at least FIGS. 9A, 9B, and 13 for further discussion.

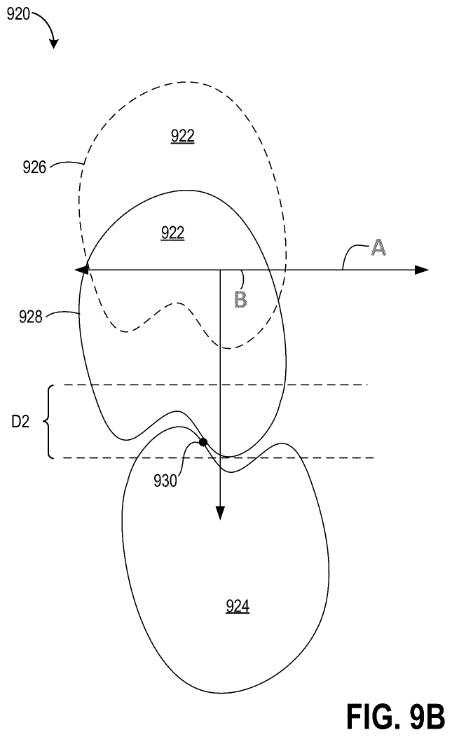

In block K (140), the computer system 102 can iteratively adjust one or more teeth in the upper and/or lower arches of the model based on the IP contact(s) and/or vertical contact movements. The computer system 102 can iteratively adjust the teeth until a threshold level of movement is achieved. Refer to at least FIGS. 11, 12, and 13 for further discussion.

Once the computer system 102 finishes auto-designing the digital denture model for the particular patient, the computer system 102 can anchor molars in the model (block L, 142). Molars can be used as anchors, especially in orthodontics, since they are hard to move through bone. When performing an auto-setup process as described herein, teeth are set up from a midline back to the molars. While aligning teeth along an arch form, the teeth may shift, thereby causing the molars to move as well. Anchoring the molars in block L (142) can include distalizing the molars, which means pushing the molars forward into the mouth in their original positions. Distalizing the molars also may cause the entire arch form to slide forward so that the molars are returned to their original positions. As a result, upper anterior teeth may, for example, be pushed forward, thereby causing a more significant overbite than desired for the patient. This overbite can be corrected automatically by the computer system 102 and/or manually by the relevant user. The user can view the overbite once the molars are anchored and determine appropriate treatment options to correct the overbite. As an illustrative example, the user and/or the computer system 102 can perform interproximal reduction, in which some of the teeth are shaved off to allow for the molars to remain in their original positions while reducing the overbite. As another illustrative example, the user and/or the computer system 102 can remove one or more teeth, such as first or second bicuspids, to maintain the molars in their original positions while reducing the overbite.

The computer system 102 can then return the digital denture model 152 for the patient in block M (144). Returning the model 152 can include storing the model in the data store 106 for later retrieval and/or use by the computer system 102 (e.g., in generating or adjusting digital denture models for the patient in the future, for iteratively training one or more machine learning models used for auto-designing the dentures as described herein) and/or the user device 108 (e.g., in viewing and modifying the model for the patient, in approving the model and sending the model with manufacturing instructions to one or more fabrication or manufacturing devices). In the example of FIG. 1, returning the model 152 includes transmitting the model 152 to the user device 108.

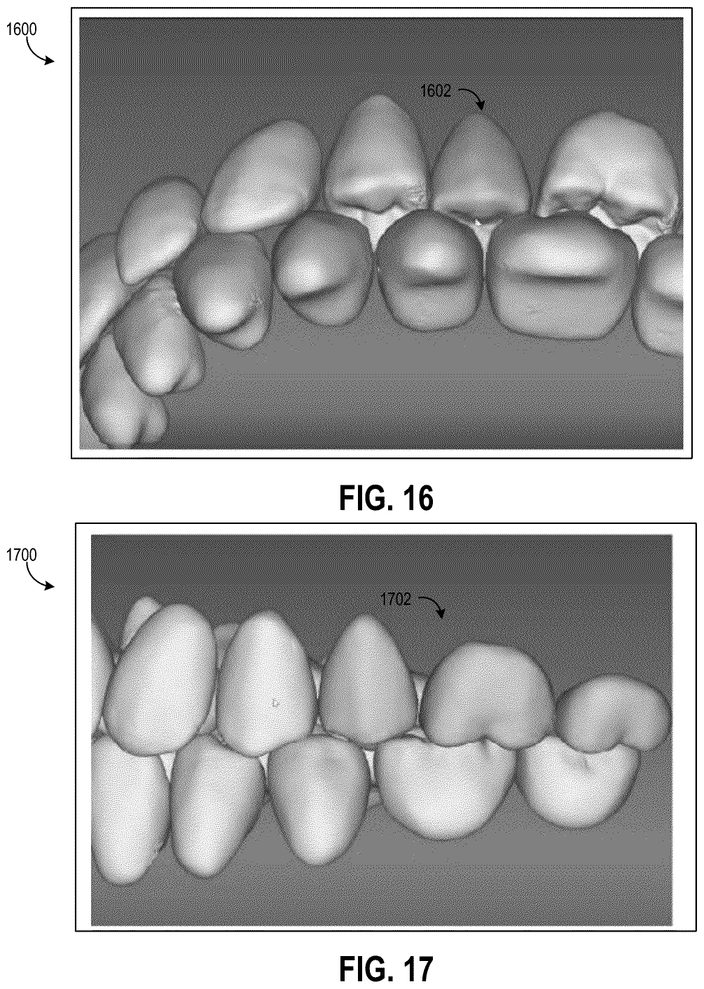

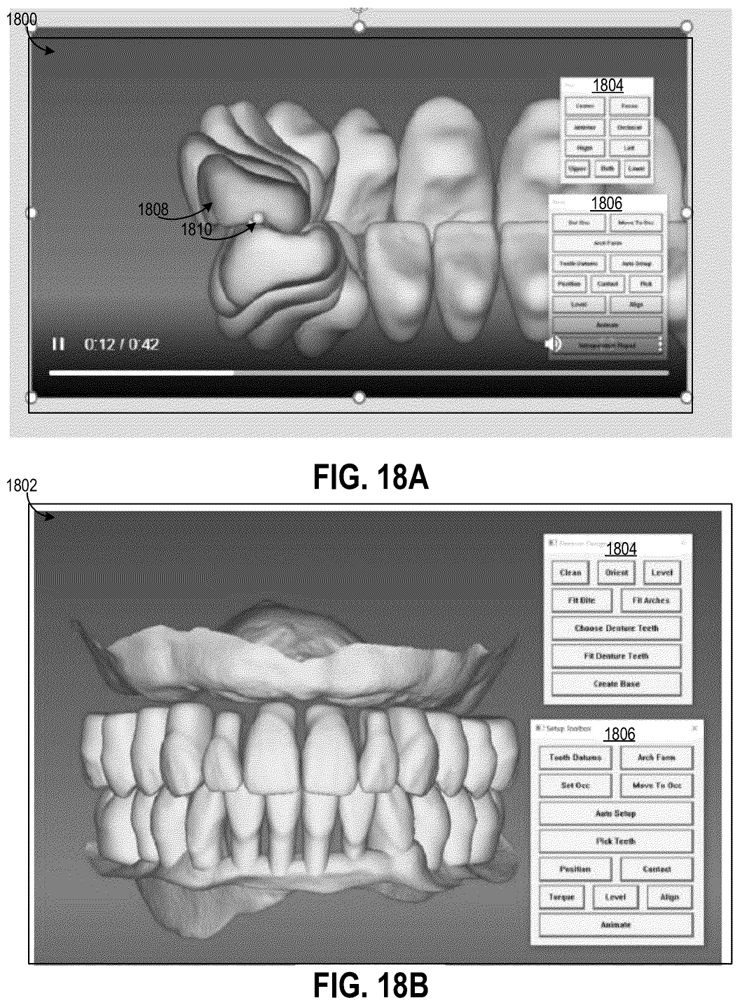

The user device 108 can output the model 152 in one or more GUIs presented in a display of the user device (block N, 146). Refer to at least FIGS. 18A, 18B, and 18C for further discussion.

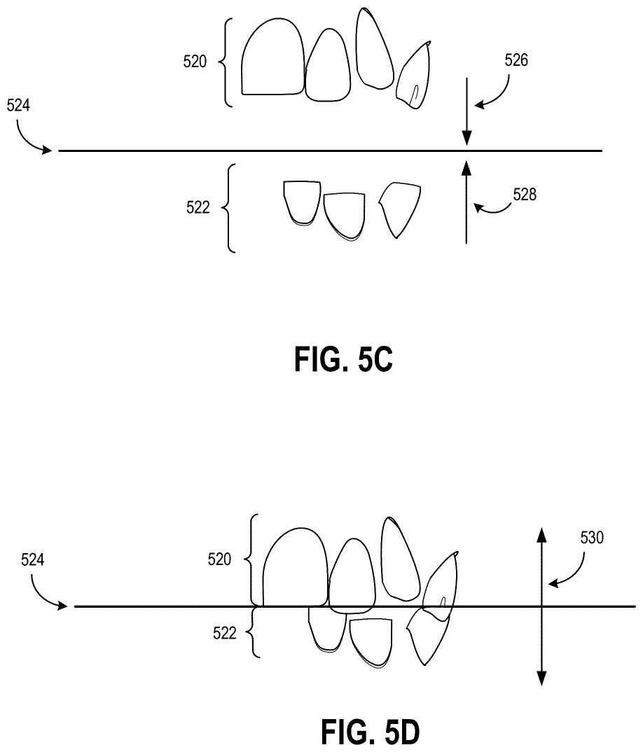

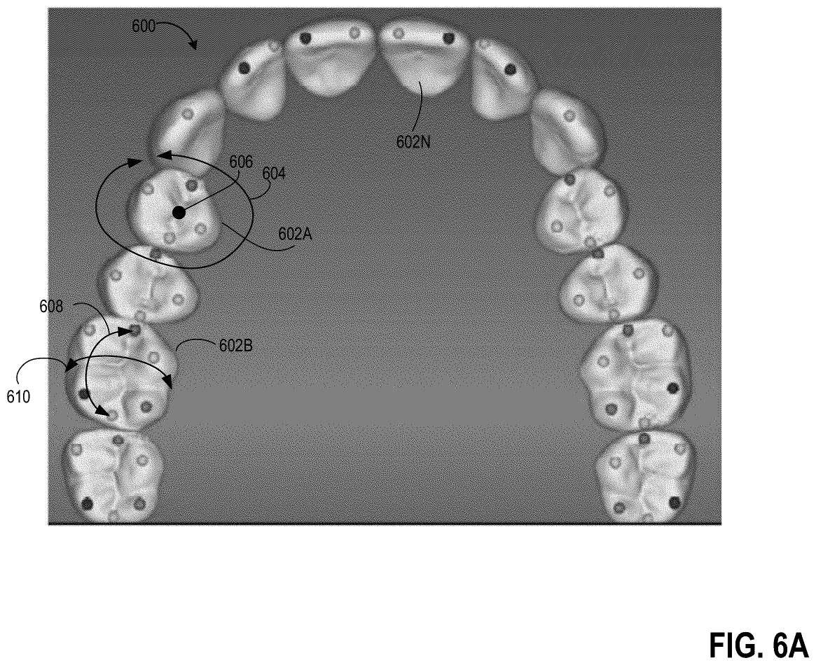

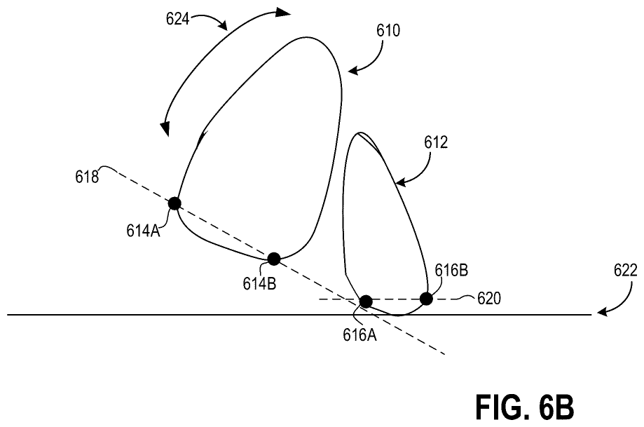

The user device 108 may optionally receive user input to modify the model 152 (block O, 148). The user input can be transmitted to the computer system 102, which can perform one or more of the blocks described above (such as blocks G-K, 132-140, respectively) to auto-adjust the teeth in the model 152 based on the user input (e.g., ensuring that when the user moves one tooth, adjacent teeth are also appropriately adjusted to avoid collision or interference). As described herein, the user can click on any one or more teeth in the model 152 and change the tooth's position and/or orientation. Once the user makes such adjustments, the user can select an option to auto-setup the teeth in the model again and/or the computer system 102 can automatically perform one or more of the auto-setup operations described herein. As an illustrative example, the user can rotate one tooth, such as a molar. Based on this user input, the computer system 102 can then automatically adjust only IP contacts and/or snap the teeth to the occlusal plane. The user can view only the lower teeth or the upper teeth of the model 152 and make adjustments to each individually. Once the user is satisfied with the arrangement of the lower teeth, for example, the user can select the upper teeth and then select an option to perform an auto-setup process for the upper teeth. Refer to at least FIGS. 6A, 6B, 6C, 16, and 17 for further discussion.

In FIG. 1, the blocks A-O, 120-148, respectively, are further described in reference to at least FIGS. 2, 3A, 3B, 3C, 10, 11, 12, 13, and 21. The adjustments made to the teeth in the model described herein can include rotating, tipping, and/or torqueing one or more teeth and/or groups of teeth. The computer system 102 can iteratively make small adjustments to the teeth to ensure that IP contacts are resolved, upper teeth are pushed down into contact with lower teeth, anterior teeth are appropriately socked to achieve a desired overbite, and posterior teeth are appropriate adjusted to achieve desired contact. The computer system 102 can then iterate through one or more of the above-described operations until a desired denture design is achieved for the particular patient.

FIG. 2 is a flowchart of an example process 200 for automatic design of a denture and/or a replacement denture for a patient. The process 200 can be performed by the digital denture design system 102 described in reference to at least FIG. 1. The process 200 can also be performed by any other type of computer system, computing device, cloud-based system, and/or network of computing systems. For illustrative purposes, the process 200 is described from the perspective of a computer system.

Referring to the process 200 in FIG. 2, at block 202, a scan of an existing denture is received by the computer system. In some implementations, the scan can be produced using a CT scanning device, a CBCT scanning device, an intraoral scanner, or a desktop scanner. In some implementations, the scan is processed by the computer system upon receipt, for example by detecting and filling in voids in the scan, removing scan artifacts arising from metallic inclusions in the dentures, correcting holes in the scan arising from positioning of the dentures in a jig or on a surface during the scan, thresholding the scan data to produce a surface scan, or converting a file type of the scan to another file type. For example, in some implementations, the scan data is converted from the native file type of the CT scanner (for example, a Digital Imaging and Communications in Medicine (DICOM) file) to a data model file type such as a stereolithography (STL) file type. In some implementations, the DICOM file or other native file of the CT scanner is converted to a polygon file format (PLY), a standard 3D image file format (OBJ), an additive manufacturing file format (AMF), a 3D manufacturing file format (3MF), or any other suitable file type.

In some implementations, additional digital patient data can also be received, for example motion data or color images of the dentures. Motion data can be captured using a motion capture system and can represent the patient's jaw moving through various jaw movements for use in preparing the replacement denture design. Color images can be obtained simultaneously with the scanning of the existing dentures and can be used to overlay color on the denture design. Additional details related to the use of motion capture data and color images can be found in U.S. Provisional Patent Application Ser. No. 63/149,178 filed on Feb. 12, 2021 and entitled “Motion-Based Digital Denture Design,” U.S. Provisional Patent Application Ser. No. 63/274,798 filed on Nov. 2, 2021 and entitled “Digital Denture Design and Replacement,” the PCT Application filed Feb. 10, 2022 and entitled “Digital Denture Design and Replacement,” and in U.S. Provisional Patent Application Ser. No. 63/313,723 filed on Feb. 24, 2022 and titled “Color Digital Denture Design and Replacement,” the contents of each of which is incorporated herein by reference in its entirety.

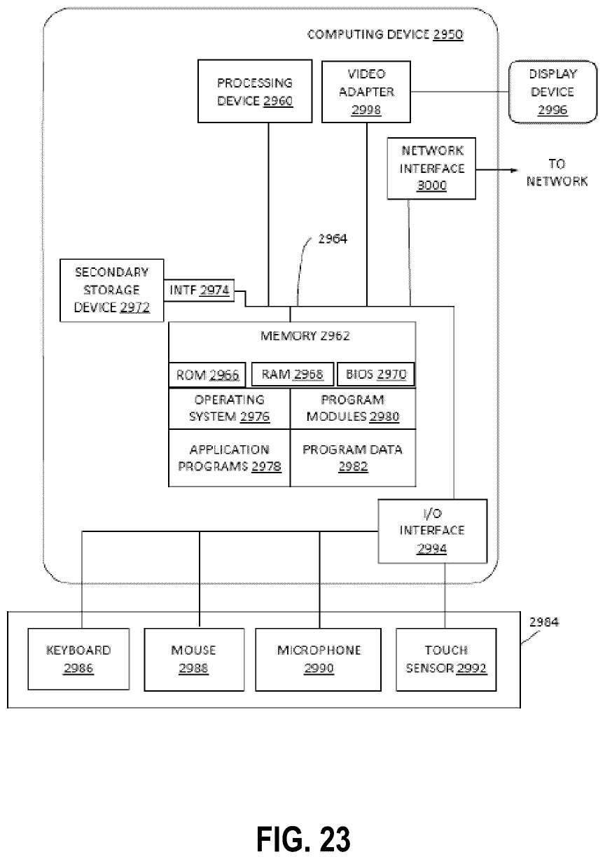

At block 204, a tooth mold can be selected by the computer system from a tooth library and based on the scan of the existing dentures. Multiple tooth libraries exist and can be stored in a static data store, which is described further in reference to FIGS. 22-23. The tooth libraries can include sets of teeth that can be chosen for inclusion in dentures and other oral prosthetics designs. These libraries can be accessed and reviewed by the computer system to select a tooth mold having a close fit to the teeth of the existing denture based on the scan or other patient tooth data. The digital denture teeth libraries may vary functionally, aesthetically, and/or based on manufacturer. The software can use a number of mechanisms for automatically selecting and scoring the match between the existing denture teeth and the tooth mold options in one or more libraries of teeth, including but not limited to a software scoring scheme that determines a perfect or preferred or best match or closest alternative from available libraries or set of libraries.

In some implementations, selection of the tooth mold is made by the computer system based on landmarks and anatomical dimensions of a subset of teeth of the denture scan and the selected tooth mold can be applied for all teeth in the denture. For example, a width of a particular tooth can be used as an initial selection criteria for determining a best matching tooth mold from the library. In some implementations, the width of one or more teeth can be used to identify a subset of candidate libraries, and an iterative fit algorithm or other mechanism can be used to determine a best or preferred library from the subset of candidate libraries. These operations can advantageously reduce an amount of processing power required for determining a best-fit library compared to using an iterative algorithm across a large number of potential libraries.

In some implementations, the selection of the tooth mold can be made for each individual tooth in the denture based on the landmark and anatomical dimensions of the particular tooth. In some implementations, the selection of the tooth mold is made based on a tooth or a subset of teeth in the upper denture, and the selected tooth mold that is the perfect or closest match is then applied to the lower denture. In some implementations, the selection of the tooth mold can be independent for the upper denture and the lower denture. The selection of the closest matching tooth mold to the existing dentures may also take into account the landmarks and dimensions of teeth on both the upper and lower dentures. Sometimes, additional information including but not limited to patient specific motion from motion capture scans and/or images and hinge axis information (e.g., from measuring jaw movement of the patient) can also be accounted for in the computer-automatic matching and selection of the tooth mold from one or more tooth libraries.

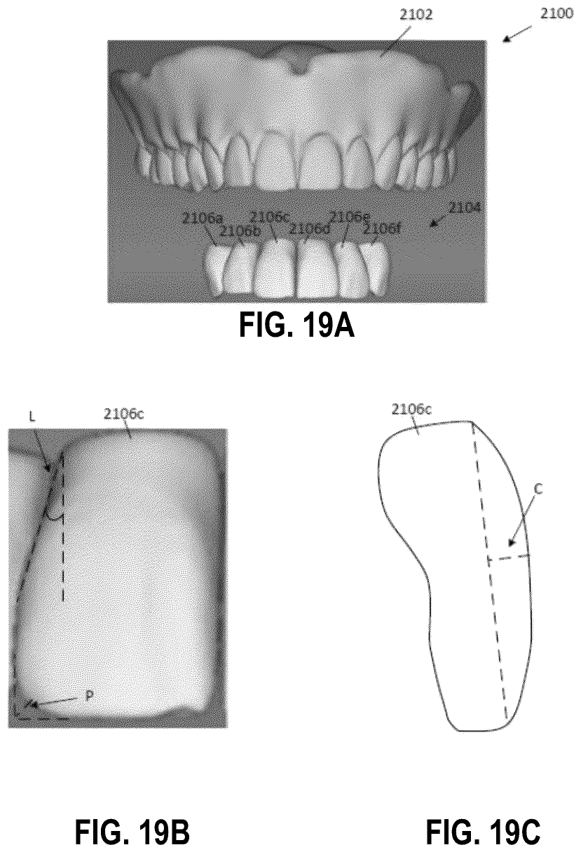

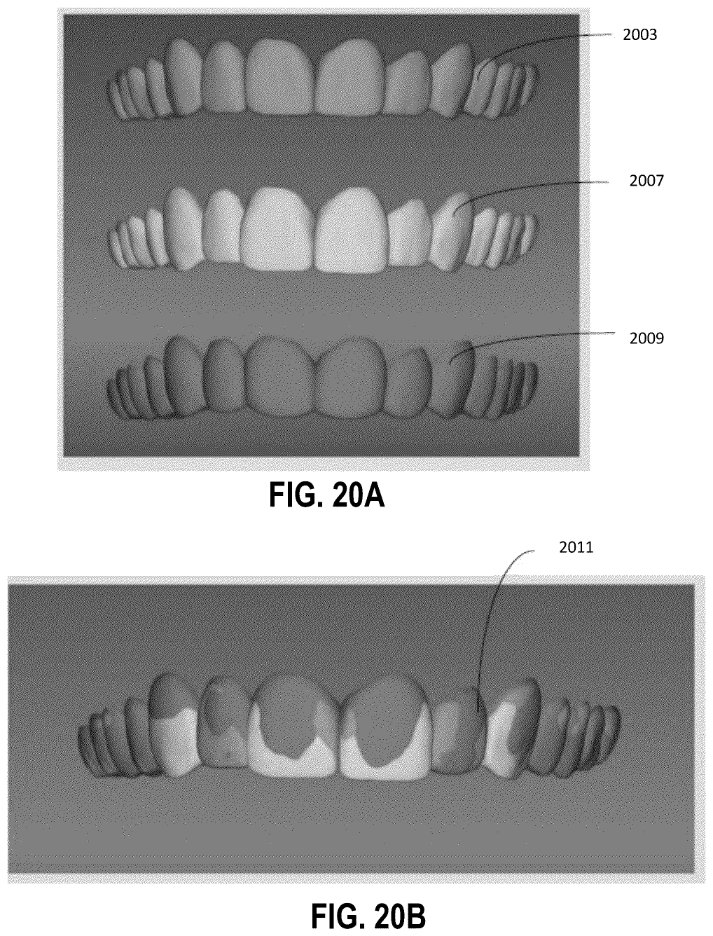

The selected denture tooth library may be displayed visually so that a user may confirm or reject the selection. The closest or perfect match can be automatically selected and presented in a GUI, as described further below. Alternatively or additionally, top matches can be presented in the GUI for user selection. In some implementations, several candidate denture tooth libraries (e.g., three that each has a closest width to that of the digital reference denture model) can be selected and presented to a user. In some implementations, the closest match can be presented with one, two, three, or four next closest matches for user confirmation or selection. A user may use a GUI to select between these options. In some implementations, the closest match and other next closest matches can be presented separately and/or in an overlay presentation using a color map to illustrate differences across the different tooth molds (for example, the separate presentation of the molds in FIG. 20A and an overlay presentation in FIG. 20B).

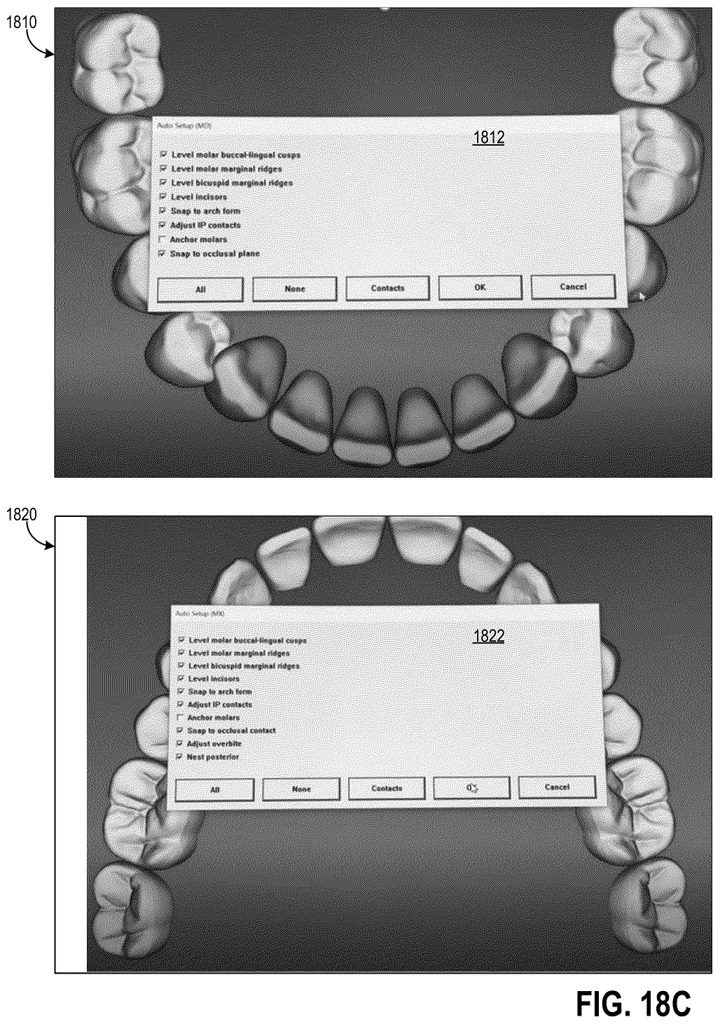

Enabling the automatic matching of denture landmarks to an existing library tooth mold can efficiently identify closest matching teeth to an existing denture. The speed with which the computer system can identify a match using an iterative fitting algorithm, such as iterative closest point (ICP) or another algorithm, is much greater than the speed with which a technician can determine an appropriate library of teeth. This process is described further below with reference to FIGS. 19A, 19B, and 19C. Accordingly, the use of an automatic selection program can address an existing backlog of replacement denture design requirements, and can also eliminate inter-technician variability to improve patient acceptance of replacement dentures and/or original dentures. The disclosed technology can further generate statistics and visualizations to quantify a fit of the tooth mold from the library to the existing dentures, and the information can be presented to a technician for approval or adjustment to patient specifications, desires, or dental requirements.

At block 206, anatomic landmarks can be identified in the scan of existing dentures by the computer system. The identification of individual teeth can be determined, for example, molars, pre-molars, canines, and incisors. Additionally, A width of each tooth or of particular teeth can be determined by the computer system. The height, circumference, or cross-section can also be determined. Other landmarks such as cusps, pits, offsets, and grooves can be identified in the scan. The identified landmarks can be presented to the technician through the GUIs described herein, as described further in reference to FIG. 22.

The GUI can include a presentation field in which the digital denture model can be displayed and manipulated by the user. The user can interact with the digital denture model by clicking and dragging, using arrow keys, using a joystick, or by using any other user selection interface. The GUIs can include a toolbox for displaying options for manipulation, selection, display, and adjustment of the digital denture model displayed in the user interface. In some implementations, the GUI can include a specialized process flow for guiding the user through the design process in a particular order. Refer to at least FIGS. 18A-C for further discussion about the GUIs.

In some implementations, the computer system can identify missing portions of a denture scan, for example if a denture tooth is broken off, the computer system identifies this as a discrepancy and can alert a user, such as the technician, through the user interface and/or suggest a tooth for positioning in the missing portion, based on selection of other teeth to fit the digital denture model and further based on the denture scan.

In yet some implementations, a process flow in the GUI can guide the user through the design and approval of a digital design of the soft-tissue adjacent portion of the dentures based on the denture scan. After the soft-tissue design process or before the soft-tissue design process, the GUI guides the user through the digital denture design process components that may include, but are not limited to, automatically selecting, fitting, and/or aligning teeth in the denture.

Still referring to FIG. 2, at block 208, the teeth from the selected tooth mold can be selected and positioned on the denture scan based on the identified landmarks and other tooth morphology. For example, a tooth from the library tooth mold can be selected by the computer system for a particular tooth of the denture scan and based on a shape matching of the teeth and/or based on a labeling of the library tooth mold teeth and labeling applied to the scan based on the identified landmarks. In some implementations, the digital denture teeth can be positioned based on a determined or selected occlusal guidance surface. In some implementations, the digital denture teeth may include labels for anatomical landmarks such as cusps, marginal ridges, incisal edges, fossa, grooves, base boundaries, and/or other anatomical landmarks. These labels may be used to automatically position the digital denture teeth with respect to one another and digital denture teeth on the opposing dentition.

The digital denture teeth may be initially positioned in alignment with an arch curve. The arch curve may be sized and shaped based on the digital dental model. Refer to the process 300 in FIGS. 3A, 3B, and 3C for additional discussion. Each of the digital denture teeth may include one or more labels that specify one or more locations on the digital denture teeth to align to the arch curve. The digital denture teeth may also be associated with a tip and torque with respect to one or more of the arch curve and the occlusal plane. When initially positioned, the digital denture teeth may be positioned based with respect to the arch curve based on the labels and automatically tipped and torqued with respect to the arch curve based on the associated values.

Once the digital denture teeth are in their initial positions, their positions may be further refined. At block 210, the teeth can be leveled and aligned to the arch form and to the occlusal plane by the computer system. Once selected, the tooth from the library tooth mold can be automatically positioned in the scan so as to most closely match the tooth in the existing denture. This can include leveling the teeth and aligning the teeth on the arch. For example, an iterative closest point (ICP) algorithm can be used to determine the appropriate position of the library tooth mold tooth to match the tooth in the existing denture. An ICP algorithm can determine not only a general “best-fit” but can determine the best-fit within microns.

After the teeth are automatically adjusted to fit the arch and the digital scan, the denture scan including the library tooth mold teeth can also be presented in the GUIs to enable the technician to manipulate and adjust the teeth. At block 212, the occlusal and proximal contacts can be adjusted by the computer system. The GUI may receive user input to move the selected digital denture tooth. In some implementations, the user input can include a drag input such as a click-and-drag or touch-and-drag. Based on a direction of the drag, the digital denture tooth may move in a corresponding direction. In some implementations, the movement may be in a direction that is parallel to the occlusal plane. In some implementations, as the digital denture tooth moves based on the drag input, the digital denture tooth also can move in an occlusal-gingival direction to make contact with the opposing dentition. In some implementations, the digital denture tooth may be moved to contact with an occlusal guidance surface that is generated based on opposing denture teeth and motion data (e.g., by sweeping the opposing denture teeth through the motion of the motion data).

Using collision informed design mechanisms, the teeth can be automatically positioned by the computer system such that adjacent teeth avoid interference or overlap with one another. The teeth can behave as real teeth while they are adjusted to the digital scan of the denture. Using real-time collision detection algorithms, the surface of the tooth can be moved with respect to other teeth in the area by the computer system. The teeth can be moved in multiple directions, including vertically and horizontally, and can be twisted or tilted to adjust the contacts and positioning. The upper denture and lower denture can also be manipulated relative to one another automatically by the computer system and/or manually by the technician or other relevant user. Individual teeth, groupings of teeth, or entire upper or lower dentures can be adjusted.

One or more teeth can be moved together by the computer system to adjust occlusal and/or proximal contacts. Using collision avoidance, the digital teeth behave like actual teeth when they are adjusted in that the teeth cannot pass through each other but are instead restricted in their movement by adjacent teeth. For example, when one tooth is selected and adjusted, it can be bounded by a tooth on either side and can only move to be adjacent the teeth but cannot move through the teeth or into the teeth. Similarly, when multiple teeth of an upper denture are selected for adjustment together, the selected teeth can maintain their original spacing but can move up or down to maintain contact with the teeth of the lower denture.

Following block 212, the denture design can be finalized by the computer system. In some implementations, the denture base can also be automatically generated in the software and presented in the user interface for approval or adjustment. In some implementations, a soft-tissue boundary curve is generated based on the digital dental model. The soft-tissue boundary curve represents the edge of the denture base. Once the soft-tissue interface is adjusted, tooth boundary curves can also be identified for each of the positioned digital denture teeth. In some implementations, the denture design is presented in the user interface for approval and can then be used to fabricate the replacement dentures.

The denture base may be fabricated based on the digital representation (e.g., the digital denture model). The denture base may be fabricated using a rapid fabrication technology such as three-dimensional printing or computer numerically controlled (CNC) milling. The denture teeth may also be manufactured using rapid fabrication technology. For example, the denture teeth may be fabricated using a three-dimensional printer or a CNC mill.

Automating the design process as described herein can increase efficiency of denture design by reducing design aspects that a technician must complete from scratch, manually, and/or visually. Providing suggestions of tooth libraries, landmark identifications, and tooth positions can reduce the amount of work and time required from a technician, thereby accelerating the process and reducing costs associated with the design of dentures. Rather than begin the design from scratch and prepare the denture design based on visual inspection of existing dentures or denture scan, the computer system can make suggestions based on advanced and complex best-fit algorithms to produce a denture design that closely matches the existing dentures in fit, function, and appearance. The high-quality denture designs from the automation of the design process can reduce the time to design and produce dentures and also reduce the cost of developing the denture design while minimizing differences in dentures designed by various technicians for improved patient experience.

FIGS. 3A, 3B, and 3C are a flowchart of a process 300 for computer-automated design (e.g., auto-setup) of dentures for a patient. The process 300 can be performed by the digital denture design computer system 102 described in reference to at least FIG. 1. The process 300 can also be performed by any other type of computer system, computing device, cloud-based system, and/or network of computing systems. For illustrative purposes, the process 300 is described from the perspective of a computer system.

Referring to the process 300 in FIGS. 3A, 3B, and 3C, the computer system can select and load library teeth for a digital denture model of a patient (block 302). Refer to at least FIGS. 1, 2, and 22 for further discussion.

The computer system can define a lower arch form for a lower portion of teeth in the digital denture model in block 304. For example, the computer system can adjust the lower arch form so that it goes through (i) a buccal side of lower anterior teeth and/or (ii) buccal cusps in lower posterior teeth (block 306). In some implementations, the computer system can receive user input from a user device of a relevant user, the user input indicating one or more adjustments to be made to the lower arch form. The arch form can be defined by the computer system and therefore used to align teeth of the digital denture model along.

In block 308, the computer system can define an upper arch form for an upper portion of teeth in the digital denture model based on the lower arch form. The computer system may optionally adjust the upper arch form so that it (i) is positioned on an inside of an upper midline of upper anterior teeth and/or (ii) goes down through center grooves of upper posterior teeth (block 310). As described herein, the upper arch form can be the same as the lower arch form. Accordingly, the computer system can mirror the lower arch form for the upper teeth. The computer system can also make some adjustments to the upper arch form once the lower arch form is mirrored/replicated for the upper teeth. In some implementations, the computer system can receive user input indicating one or modifications to shape and/or placement of the upper arch form.

The computer system can also define an occlusal plane in block 312. For example, the computer system can define a location, height, angle, cant, and/or tip of the occlusal plane relative the teeth in the digital denture model (block 314). As another example, the computer system can adjust a position of the occlusal plane relative to the lower arch (or optionally the upper arch) (block 316). The computer system can move the occlusal plane up and/or down until (for example) tips of the lower anterior teeth touch and/or slightly pass through the occlusal plane. Sometimes, a curve of Wilson (from an anterior view) and/or a curve of spee (from a side view) can be used by the computer system to curve the occlusal plane. For example, the computer system can define the occlusal plane to curve upward from the anterior view such that buccal cusp tips may be lower than lingual cusp tips. In some implementations, the computer system can receive user input indicating one or more adjustments to the occlusal plane.

In block 318, the computer system can identify and label at least one datum per tooth in the digital denture model. Refer to at least FIG. 4 for further discussion. In some implementations, the computer system can apply one or more machine learning models to the digital denture model. A model can, for example, be trained to identify and label a plurality of different types of datums, landmarks, or other types of markers for different type of teeth in the digital denture model. The computer system can also use an automated algorithm for identifying the plurality of datums in the digital denture model. The user can then provide user input to adjust placement of one or more of the identified datums. The computer system can identify and label datums that include but may not be limited to edges of cusp tips, marginal ridge points, molars, low spots between cusps where adjacent teeth may touch, canine cusp tips, etc.

As an illustrative example, the computer system can apply an algorithm or a model that has been trained to identify datums on incisors in the model. The incisor datums can include one or more datums on each incisal edge (e.g., 2 datums on an incisal edge). A biting surface of the incisor can, for example, have 2 datums. An another example, the computer system can identify at least one datum on a cusp tip for each canine in the model. For bicuspids and molars, the computer system can identify at least one datum on each cusp tip and/or marginal ridge point per bicuspid and molar. Various other datums can be identified and labeled by the computer system, as described throughout this disclosure.

The computer system can level each tooth in the digital denture model based on the datums and relative to the occlusal plane (block 320). Refer to FIGS. 5-6 and 10 for further discussion. For example, the computer system can optionally rotate the tooth around a line perpendicular through marginal ridge datums of the tooth (block 322). Additionally or alternatively, the computer system can optionally torque the tooth using buccal and/or distal cusp tip datums of the tooth (block 324). Additionally or alternatively, the computer system can optionally tip the tooth mesially and/or distally using marginal ridge datums of the tooth (block 326).

As an illustrative example, the computer system can use 2 datums on each incisor to level the respective incisor. The computer system can rotate the incisor so that it is level to the occlusal plane. In other words, the computer system can move the incisor until a tip of the incisor is level with the occlusal plane. Typically, the computer system may tip the incisor but may not torque the incisor. The computer system can perform a similar process for leveling the posterior teeth with the occlusal plane. For example, the computer system can select one tooth at a time and rotate any of the posterior teeth from a facial view of the model such that one or more marginal ridges of the posterior teeth are level with the occlusal plane and/or come into contact with the occlusal plane.

The computer system can level each tooth independently of other teeth to the occlusal plane by identifying a pivot point for the tooth. The computer system can identify a midpoint between 2 datums for the tooth. The computer system can define a line perpendicular to a line drawn through the 2 datums of the tooth (when looking down at the tooth from a top-down view) and rotate the tooth around the perpendicular line so that the 2 datums (e.g., marginal ridge points) of the tooth remain a same distance away from the occlusal plane.

Optionally, the computer system can torque (e.g., move inward and outward) at least one tooth based on the tooth's angle relative to the occlusal plane. To torque the tooth, the computer system can use buccal and/or distal cusp tip datums so that the cusp tips of the tooth are parallel to the occlusal plane (e.g., so that cusp tips of a molar are parallel to the plane). After the tooth is torqued, the computer system may also tip the tooth. For example, the computer system can tip a posterior tooth using the marginal ridge datums for the tooth. Tipping a tooth can cause a root of the tooth to move mesially and/or distally from a facial view of the tooth. In some implementations, by default, the posterior teeth can be automatically torqued and tipped. In some implementations, the user can select whether they desire for the posterior teeth (and other teeth in the model) to be torqued, tipped, and/or rotated.

In yet some implementations, specified angles for tipping and/or torqueing can be provided to the computer system in block 320. The specified angles can be provided by the user at the user device. The specified angles can additionally or alternatively be retrieved from a data store by the computer system. In orthodontics, for example, an angle of a tooth relative to the occlusal plane can be predetermined and used by the computer system when leveling the teeth. As a result, the computer system can level (e.g., torque and/or tip) the teeth until the specified angle between the teeth and the occlusal plane is achieved.

Sometimes, to tip a tooth, the computer system can identify a midpoint of a crown of the tooth, not counting a root part of the tooth. The computer system can then find all triangles within a neighborhood of the midpoint (e.g., 1 mm of the midpoint) and sum (e.g., aggregate) all the triangle surface normals (which typically can be unit vectors, such as 1 unit long) to determine an average surface normal vector for the tooth (e.g., a vector protruding straight out of the surface). The computer system can then use the average surface normal vector to determine how much to tip the tooth.

In some implementations, the tooth libraries that are loaded into the digital denture model can include a predefined coordinate system per tooth. As a result, during real-time performance of the process 300, the computer system may not have to identify all the datums and then build a coordinate system per tooth to appropriately and accurately perform the leveling, tipping, and/or torqueing described here. Instead, the retrieved tooth libraries can include stored coordinate systems, which cant hen be used by the computer system in block 320 to quickly, efficiently, and accurately level each tooth relative to the occlusal plane.

The computer system can snap the lower and/or upper teeth, individually and/or in sets/groups, to a same arch form in block 328. Refer to FIGS. 7A, 7B, 7C, and 11 for further discussion. As described herein, the same arch form can be used for both upper and lower teeth. In orthodontics, the computer system can start with snapping the lower teeth to the lower arch form because there can be limited space for movement in the lower arch compared to the upper arch (e.g., the lower arch can have less bone in a jaw to work with than the upper arch). In some implementations, it does not matter whether the lower teeth are snapped to the lower arch form before snapping the upper teeth to the upper arch form or vice-versa. Sometimes, when teeth are snapped to the arch form, the teeth may overlap or otherwise collide. Adjustments can be made by the computer system at a later time to resolve any of these collisions. The purpose of snapping the teeth to the arch form is to ensure that the teeth are appropriately tipped and torqued to achieve a desired denture design.

The computer system can adjust all or a set of IP contacts in the digital denture model to remove tooth overlap and/or maintain relative tooth orientation (block 330). Refer to FIGS. 8 and 12 for further discussion. For example, the computer system can solve for IP contacts by starting at a midline of anterior teeth and working back towards molars on each side of a respective arch form. Resolving the IP contacts can provide for resolving any interference problems between neighboring teeth. Resolving the IP contacts can be performed in 2D. Therefore, the previously determined tooth tips and/or torques may be locked in and the computer system may not tip and/or torque the tooth in block 330. As described further in reference to FIGS. 8 and 12, a movement direction for resolving the IP contacts can be defined by centers of at least 2 neighboring teeth and a vector that passes through the centers of the neighboring teeth. The computer system can accordingly move each tooth along the vector in order to maintain a relationship between the neighboring teeth.

The computer system can adjust vertical positioning of each tooth in the upper and/or lower arches in block 332. Refer to FIGS. 9A, 9B, and 13 for further discussion. For example, the computer system can push each lower tooth in a direction perpendicular to the occlusal plane until the lower tooth contacts the plane (block 334). Regarding the lower tooth, the computer system can keep moving the tooth until an occlusal point of the tooth reaches the occlusal plane (e.g., a point of each tooth that's closest to the occlusal plane gets pushed into contact with the occlusal plane when the occlusal plane is normal, such as when a Z coordinate of the occlusal plane is completely flat).

Additionally or alternatively, the computer system can sock each upper tooth until the upper tooth contacts one or more of the lower teeth (block 336). As described further in reference to at least FIG. 13, the computer system can push the lower teeth down until they come into contact with the lower teeth. Some teeth, such as canines of the upper teeth, may poke through the occlusal plane.

In block 338, the computer system can optionally iteratively adjust at least one upper tooth in, out, down, and/or up until a threshold distance for a predetermined overbite is achieved. Refer to FIGS. 9A and 9B for further discussion. The computer system can iteratively adjust each tooth independently of other teeth (e.g., perform the iterative adjustments on a tooth-by-tooth basis). The threshold distance can vary based on the tooth/teeth being adjusted. For example, the threshold distance can be approximately 2 mm of overbite for central teeth and incisor teeth. As another example, the threshold distance can be approximately 1.5 mm for lateral teeth. The threshold distance can be set and/or determined by the computer system. In some implementations, the user can provide user input indicating one or more desired threshold distances, which can then be used by the computer system in block 338.

In block 338, the computer system can perform one or more iterative adjustments to nest and/or sock the upper posterior teeth. For example, for each upper posterior tooth, the computer system can move the tooth in and out, buccal-lingually, until a lowest point that the tooth can go is achieved (or some predetermined threshold distance is achieved). If, as an illustrative example, the tooth is moved in a buccal direction that causes the tooth to also move up, then the computer system can determine a distance from the tooth to one or more lower teeth (and/or the occlusal plane), divide the distance in half, and move the tooth in the opposite direction (e.g., lingually) by an amount the corresponds to half the distance. If the tooth is moved in the buccal direction and the tooth continues to move down, then the computer system can continue to move the tooth in the buccal direction until the desired lowest point/predetermined threshold distance is achieved.

The computer system can determine, in block 340, whether positioning of the teeth in the digital denture model achieves one or more denture design criteria. If the one or more denture design criteria is not achieved, the computer system can return to block 330 in the process 300 and repeat the blocks 330-340 until the one or more denture design criteria is achieved. For example, the computer system can iteratively adjust IP and/or vertical contacts between the upper and lower teeth until an amount of movement of the teeth satisfies a threshold amount of movement (e.g., approximately 10 microns of movement). In other words, iterative adjustments can be made to the upper and lower teeth until such adjustments cause no more than the threshold amount of movement to other teeth in the digital denture model.

If the one or more denture design criteria is achieved, the computer system can proceed to block 342, in which the computer system can anchor at least one tooth in the digital denture model. For example, the computer system can anchor molars in the model.

The computer system can return the digital denture model for presentation in a GUI at a user device of a relevant user in block 344, as described throughout this disclosure. In some implementations, the process 300 can stop at the block 344. In some implementations, the process can continue with block 346.

The computer system can optionally receive user input indicating one or more adjustments to the digital denture model (block 346), as described throughout this disclosure.

Accordingly, the computer system can automatically adjust the digital denture model based at least in part on the user input in block 348. Block 348 can optionally be performed, for example, in response to receiving user input indicating a desire to perform the auto-setup process again (or a portion of the auto-setup process). Automatically adjusting the model can include returning to block 330 in the process 300 and iterating through one or more of the blocks 330-340 until a desired design for the dentures is achieved.

Once the computer system adjusts the digital denture model in block 348, the computer system can optionally return the adjusted digital denture model, such as by presenting the adjusted model in the GUI at the user device (block 350). The process 300 can then stop. The computer system can also iterate through one or more other operations in the process 300, such as receiving additional adjustments from the user device and/or performing one or additional operations in the auto-setup process described herein.

FIG. 4 is an example user interface screen (GUI 400) that may be generated by the digital denture design system 102 described herein with labels 402A-N on identified landmarks (e.g., datums) of teeth 404 in a digital denture model 406. In this example, the labels 402A-N are displayed as spherical markers overlayed on the digital denture teeth 404 at locations of the corresponding anatomical landmarks. Different types of anatomical landmarks may be shown with different visual characteristics. Here, different types of anatomical landmarks are shaded differently (e.g., using different colors or shading characteristics). In some implementations, different types of anatomical landmarks may be shown using different textures, patterns, and/or indicia.

In this example, different label characteristics are used for the mesial-labial cusp tips (or mesial end of the incisal edge, depending on the tooth), distal-labial cusp tips (or distal end of the incisal edge, depending on the tooth), the mesial-lingual cusp tips, the distal-lingual cusp tips, the mesial end of the central fossa, and the distal end of the central fossa. Different colors, patterns, shapes, or other indicia can be used to show and label each of the characteristics of the teeth. For example, for an anterior tooth, the tooth has an incisal edge with 2 datums-1 on a mesial side closes to a midline and 1 on a distal side. The datums on the mesial (e.g., 402A) and distal (e.g., 402C) sides can be represented in different colors or indicia so that at a glance, the relevant user can easily and quickly tell the different sides of the tooth apart from each other. The same or similar coloring/indicia conventions can be applied to posterior teeth on buccal cusps. As another example, posterior teeth can include 4 datums on cusps and 2 datums on marginal ridge points. These 6 datums can be color-coded to help the relevant user easily and quickly identify each point on the tooth and/or mesial and distal sides of the tooth.

FIG. 5A is an example GUI 500 that may be generated by a digital denture design system for positioning of an occlusal plane 502 relative to a digital denture model 504. In some implementations, the occlusal plane 502 is also determined for positioning of the scan of the existing dentures. The digital denture model 504 determined from the scan as described herein can be displayed in the GUI 500. The GUI 500 may be configured to receive user input to adjust a vertical dimension of occlusion and/or a position of the occlusal plane 502. For example, the GUI 500 may be configured to receive a drag (e.g., click-and-drag or touch-and-drag) input to interactively move a mandibular arch of the digital dental model 504 up or down along an arch defined by motion data or a hinge axis inferred from the motion data. Similarly, the GUI 500 may be configured to interactively move the occlusal plane 502 along the arch between the mandibular arch and maxillary arch of the digital dental model 504, as shown by arrow 506.

A suggested position of the occlusal plane 502 can be determined by the computer system as described herein and presented to the technician in the GUI 500 for further adjustment and/or approval. In this example, the occlusal plane 502 is highlighted, indicating that it is selected and that the user may provide input to reposition the occlusal plane 502. In some implementations, the occlusal plane 502 can be visually presented in other indicia (e.g., color, pattern, glow effect, etc.) to indicate that is has been selected and that the user can manipulate/adjust it. The GUI 500 may be configured to accept one or more inputs (e.g., a button, menu actuation, scroll, drag, click) to cause the digital denture teeth 504 to move (or snap) to the occlusal plane 502. In at least some implementations, the occlusal plane may be displayed with respect to one arch, while digital denture teeth of the other arch move with the occlusal plane 502.

FIG. 5B is an example GUI 510 that illustrates adjustments made to the occlusal plane 502 relative to lower teeth 512 in a digital denture model of FIG. 5A. The occlusal plane 502 can be moved up and down to be parallel to and/or touch a cusp tip 514 of at least one of the lower teeth 512. The computer system described herein can automatically move the occlusal plane 502 to come into contact with the cusp tip 514 of at least one of the lower teeth 512. In some implementations, the GUI 510 can be presented at a user device described herein and the user device can receive user input indicating movement of the occlusal plane 502 relative to the lower teeth 512. For example, the user can use a mouse wheel to scroll the occlusal plane 502 down into contact with the cusp tip 514 of at least one of the lower teeth 512. As described above, the computer system and/or the user can also adjust the occlusal plane 502 by canting, tipping, and/or curving the occlusal plane 502 relative to the lower teeth 512.

In some implementations, the occlusal plane can be adjusted relative to the upper teeth. Sometimes, the lower teeth's occlusal plane can be adjusted to be 2 mm above the occlusal plane of the upper teeth. The teeth may only be snapped to the occlusal plane of the lower teeth, in some implementations. However, the user can also provide inputs at their device to snap teeth to the occlusal plane of the upper teeth. Adjusting the occlusal plane to the lower teeth can advantageously provide an aesthetically pleasing appearance for the patient's smile since the lower teeth can be aligned on a common plane and the upper teeth can then be socked based on the occlusal plane of the lower teeth.

FIG. 5C illustrates example teeth 520 and 522 in a digital denture model before being adjusted relative to an occlusal plane 524 for the model. FIG. 5D illustrates the example teeth 520 and 522 of FIG. 5C after being adjusted relative to the occlusal plane 524. Referring to FIG. 5C, the upper teeth 520 and the lower teeth 522 are shown relative to the occlusal plane 524 that has already been defined for a particular patient's digital denture model. The computer system described herein can automatically lower the upper teeth 520 in a direction towards the occlusal plane 524 as shown by an arrow 526. The computer system can automatically raise the lower teeth 522 in a direction towards the occlusal plane 524 as shown by an arrow 528. In some implementations, the upper and/or lower teeth 520 and 522, respectively, can be displayed in a GUI at a user device with the occlusal plane 524. The user device can then receive user input indicating one or more movements of the upper and/or lower teeth 520 and 522 closer and/or farther away from the occlusal plane.

Referring to FIG. 5D, the upper teeth 520 have been moved down so that a tip of at least one of the upper teeth 520 is in contact with the occlusal plane 524. The lower teeth 522 have also been raised so that a tip of at least one of the lower teeth 522 comes into contact with the occlusal plane 524. In some implementations, the upper teeth 520 can be lowered until at least one of the upper teeth 520 comes into contact with at least one of the lower teeth 522. In some implementations, as described herein, the occlusal plane 524 can be defined relative to the lower teeth 522, the lower teeth 522 can optionally be moved up to come into contact with the occlusal plane 524, and then the upper teeth can be moved down to come into contact with either the occlusal plane 524 or at least one of the lower teeth 522. The upper teeth 520 and the lower teeth 522 can be moved, independently of each other or together, relative to the occlusal plane 524 in up and/or down directions, as shown by an arrow 530 in FIG. 5D. Furthermore, the teeth 520 and 522 shown in FIGS. 5C and 5D have not yet been rotated, straightened, snapped, tipped, torqued, or otherwise adjusted by the computer system or the user as described throughout this disclosure. One or more of these auto-setup operations can be performed after the teeth 520 and 522 are adjusted/leveled with the occlusal plane 524.

FIG. 6A illustrates example adjustments of rotating 604, tipping 608, and/or torqueing 610 one or more teeth 602A-N in a digital denture model 600. The rotating 604, tipping 608, and/or torqueing 610 can be performed automatically by the computer system described herein and/or manually by a relevant user at their respective user device. Refer to blocks 320-326 in the process 300 in FIG. 3B for further discussion about rotating, tipping, and/or torqueing teeth. Although rotating, tipping, and torqueing are described and depicted in reference to lower teeth in the model 600, the same or similar techniques can also be applied to upper teeth in the model 600.

In the example of FIG. 6A, the tooth 602A is rotated around a pivot point 606. The pivot point 606 can be defined as a midpoint between 2 datums of the tooth 602A. The pivot point can be identified as a valley between one or more marginal ridges in the tooth 602A. In some implementations, the pivot point can be either one of the datums described herein. When looking down at the tooth 602A, the relevant user can click on, as an example, one of the mesial datums, drag it over to an arch form curve for the digital denture model 600, and then pivot the tooth 602A about that datum to cause the other mesial datum to come into contact with the arch form curve.

The tooth 602B can be tipped 608 and/or torqued 610. When the tooth 602B is tipped, the tooth 602B's orientation is being changed. For example, the tooth 602B can be rotated laterally (e.g., around a pivot point) when viewing the tooth 602B from a facial (e.g., buccal, labial) view. From the facial view, the tooth 602B can be rotated about a vector coming off a face of the tooth 602B and directed towards a viewer of the tooth. In some implementations, the tooth 602B can be tipped relative to its contact points with one or more adjacent teeth in the model 600. In some implementations, only anterior teeth in the model 600, such as the tooth 602N, may be tipped using the disclosed techniques.

When the tooth 602B is torqued, a root of the tooth 602B can be rotated in and out (e.g., in and out of a mouth, or buccal-lingually). The torque adjustments can impact how far embedded the root of the tooth 602B is inside the patient's mouth. Posterior teeth in the model 600, such as molars, can be torqued. In some implementations, one or more of the anterior teeth, such as the tooth 602N, can be torqued. An axes of rotation for a posterior or anterior tooth can be a vector parallel to a reference line that passes through proximal contacts (e.g., where 2 neighboring or adjacent teeth touch each other) of the tooth. In some implementations, the axes of rotation can be a vector that is parallel to a reference line that passes through marginal ridges of the tooth. A pivot point for the tooth can be sub-gingival, meaning somewhere inside the patient's gum, thereby causing the tooth/root to be moved in and out of the gums of the patient's mouth. Although the proximal contacts, which are in a top ⅔ of the tooth, may be used to determine the axes of rotation, the actual axes of rotation can be lower along the tooth so that the actual movements/torqueing occurs down inside the gums (e.g., sub-gingival).

FIG. 6B illustrates example tipping adjustments that can be made to teeth 610 and 612 in a digital denture model. In FIG. 6B, the teeth 610 and 612 are adjacent upper teeth. The teeth 610 and 612 are presented with an occlusal plane 622 from a front-facing view. The tooth 610 can be tipped laterally, as shown by an arrow 524, without adjusting vertical positioning of the tooth 610. The tooth 610 can be tipped, by the computer system and/or by a user as described herein, until at least a portion of the tooth 610 comes into contact with a portion of the tooth 612.

Pivot points 614A and 614B can be defined for the tooth 610. A line 618 can be defined to pass through the pivot points 614A and 614B. The line 618 can be used as a reference point to tip the tooth 610 as shown by the arrow 624. For example, the computer system can tip the tooth 610 until the line 618 for the tooth 610 is parallel to/with the occlusal plane 622. Once the line 618 is parallel to/with the plane 622, the computer system can stop tipping the tooth 610. In some implementations, the tooth 610′s line 618 may be parallel to/with the plane 622 but the tooth 610 may not be touching or otherwise contacting the tooth 612. Additional adjustments can then be made by the computer system to adjust contacts/interferences between the tooth 610 and the tooth 612.

Similarly, pivot points 616A and 616B can be defined for the tooth 610. A line 620 can be defined to pass through the pivot points 616A and 616B. The line 620 can also be used as a reference for tipping the tooth 612. In the example of FIG. 6B, the line 620 is already parallel to/with the occlusal plane 622. Therefore, the computer system can determine that the tooth 612 does not need to be tipped; only the tooth 610 may be tipped.

FIG. 6C illustrates example torqueing adjustments that can be made to teeth in a digital denture model 630. The computer system described herein can provide an anterior view of both upper and lower arches 640 and 650, respectively, of the digital denture model 630. An initial auto-setup of teeth in the upper and lower arches 640 and 650 can be performed by the computer system and presented in a GUI at a user device, as described herein. A relevant user can interactively adjust any one or more of the teeth in the upper and/or lower arches 640 and 650.

For example, the user can click on/select a posterior tooth 632. The selected tooth 632 can be visually presented in a shading, color, highlighting, pattern, or other type of indicia that is different than a visual appearance of other teeth in the upper and lower arches 640 and 650. Here, the selected posterior tooth 632 is presented in a blue color. Once the tooth 632 is selected, a pivot point 634 can be identified by the computer system and visually presented as overlaying a portion of the tooth 632. In the example of FIG. 6C, the pivot point 634 visually overlays a midpoint of the tooth 632. The tooth 632 can be torqued according to the pivot point 634. The tooth 632 can be torqued in a direction shown by an arrow 636.

To manually adjust the torque of the tooth 632, the user can click and drag the tooth 632. The user can also select one or more teeth in a group to adjust the group as a whole. For example, the user can select all the posterior teeth in the upper arch 640 and adjust a torque for the group of posterior teeth.

Once the user and/or the computer system adjusts the torque of one or more teeth, the computer system can perform another iteration of one or more auto-setup operations (e.g., for just the upper arch of teeth, for just the lower arch of teeth, or for both arches) to ensure that all teeth are aligned and positioned appropriately based on the user modifications. For example, after the user adjusts the torque of the posterior tooth 632, the auto-setup operations performed thereafter can include auto-adjustments to posterior teeth in contact with the user-torqued posterior tooth 632, such as adjusting the IP contacts, snapping to the occlusal contact, adjusting overbite, and/or nesting.

FIG. 7A is an example GUI 700 that may be generated by the digital denture design system for identifying an arch 702 of a digital denture model 704. The GUI 700 shows the example arch curve 702, which can be used in positioning the library tooth mold teeth on the digital denture model 704 produced from the denture scan. The example arch 702 may be used to initially position the digital denture teeth. Here, the arch curve 702 is a spline curve shown with control points 706A-N. The control points 706A-N can be shown as spheres. The control points 706A-N can also be visually depicted in any other variety of ways. In some implementations, the GUI 700 accepts inputs to change positions of one or more of the control points 706A-N (e.g., a drag may reposition a selected control point) and causes the computer system described herein to adjust a shape of the arch curve 702 accordingly. In some implementations, the digital denture teeth are repositioned as the arch curve 702 changes. In some implementations, the arch curve 702 may be adjusted independently of the digital denture teeth. The GUI 700 may be configured to accept one or more inputs (e.g., a button or menu actuation) to cause the digital denture teeth to re-align to the arch curve 702. In some implementations, adjusting control points 706A-N on one side of the arch curve 702 can cause the computer system described herein to mimic the control point adjustments on an opposite side of the arch curve 702 to provide a uniform arch form for the denture model 704. In some implementations, adjustments can be made to only one side of the arch curve 702.

FIG. 7B is an example GUI 710 that may be generated by the digital denture design system for providing options to a technician to aid in adjusting an arch of a digital denture model. FIG. 7B shows the GUI of FIG. 7A with an example dialog box 712 to allow a user to adjust settings to automatically setup (position) digital denture teeth. The dialog box 712 has checkboxes to control how the digital denture teeth are automatically positioned by the computer system described herein and/or relative to the arch form. The user can select one or more of the checkboxes in the dialog box 712 to reposition the denture teeth. In some implementations, the digital denture teeth may be aligned to the occlusal guidance surface. For example, the cusp tips and incisal edges may be aligned to the occlusal guidance surface. As described previously, the digital denture teeth of one of the arches (e.g., the lower arch) may be positioned based on one or more of an arch curve, an occlusal plane, or an occlusal guidance surface. The digital denture teeth of the other arch (e.g., the upper arch) may then be positioned based on the positions of the digital denture teeth of the first arch.

In some implementations, the selected teeth from the library tooth mold can be down-sampled prior to positioning on the denture scan. For example, an identified subset of candidate libraries can be down-sampled to a few thousand points for each library in order to speed up the process of determining the best-fit. While high-fidelity images are often used for manufacturing of the teeth and dentures, for the design process a lower-fidelity model is often sufficient, and the down-sampled tooth libraries can significantly reduce the processing power and time required for the automatic selection and positioning of the teeth. Tooth molds available from tooth libraries can in some cases be very high-resolution digital models, which require large amounts of processing power to manipulate or compare to lower resolution denture scans. By down-sampling the digital models of the tooth molds prior to manipulation in the software using the computer system described herein and display in the user interface, the processing can be more efficiently managed without impacting the produced denture design. In some implementations, multiple options for resolution can be selected by a technician to determine the resolution of the denture design output.

The dialog box 712 can include one or more additional and/or alternative selections for automatically setting up the digital denture teeth. For example, the dialog box 712 can include selectable options to: level molar marginal ridges, level bicuspid marginal ridges, level incisors, and/or anchor molars. In some implementations, default auto-adjustment settings may include selection of options to: level molar buccal-lingual cusps, level molar marginal ridges, level incisors, snap to arch form, adjust IP contacts, and snap to occlusal plane. One or more other default settings can be applied. As described herein, the user can also select or deselect any of the options presented in the dialog box 712 to indicate what aspects of the digital denture teeth the user desires to be auto-setup by the computer system described herein.

FIG. 7C illustrates snapping one or more lower teeth to the arch 702 of a digital denture model. In the example of FIG. 7C, lower teeth 720 are shown with the arch 702 overlaying a portion of the lower teeth 720. A lower tooth 722 of the lower teeth 720 can be snapped to the arch 702 by the computer system and using the disclosed techniques. For example, 2 points 724A and 724B on the tooth 722 can be identified by the computer system. The points 724A and 724B can be datums that were previously identified by the computer system, as described at least in reference to the process 300 in FIGS. 3A, 3B, and 3C. The points 724A and 724B can be opposing cusp tips or edges on the tooth 722. A reference line 726 can be identified to pass through the points 724A and 724B of the tooth 722. The tooth 722 can then be snapped by the computer system by rotating the tooth 722 until the reference line 726 is parallel with a tangent line 728. The line 728 can be tangent to a curve of the arch 702. Additionally or alternatively, snapping the tooth 722 to the arch 702 can include moving the tooth 722 in and out (e.g., buccal-lingually) in a direction illustrated by an arrow 730. The tooth 722 can be moved in and out until a desired distance D is achieved between the reference line 726 and the tangent line 728.

The operations described herein to snap the tooth 722 to the arch 702 can also be performed to snap the other teeth in the digital denture model to the arch 702. In other words, the computer system can snap each tooth to the arch 702 on an individual, tooth-by-tooth basis. In some implementations, the computer system can snap a group or set of teeth to the arch 702 at a time. The computer system can also snap upper teeth separately from the lower teeth 720. For example, the computer system can snap the lower teeth 720 by moving the lower teeth 720 to be on, or otherwise have a buccal or facial surface of the lower teeth 720, touching the tangent line 728. The computer system can snap the upper teeth by moving the upper teeth to be positioned a predetermined distance beyond the tangent line 728. In other words, the upper teeth can be positioned so that a buccal or facial surface of the upper teeth pass the tangent line 728 and are positioned closer to the patient's cheek rather than the patient's tongue. Such snapping adjustments can be made to achieve a desired overbite for the patient.

FIG. 7D illustrates an arch form 760 for both upper and lower teeth 750 and 740, respectively, of a digital denture model. As described herein, the same arch form 760 can be used for both the upper teeth 750 and the lower teeth 740. A user, as described herein, can click, select, and/or drag any points 762A-N to adjust the arch form 760. When the user moves one of the points 762A-N in the arch form 760 of the lower teeth 740 a same adjustment can be replicated, by the computer system, in the arch form 760 of the upper teeth 750. In some implementations, the user can select an option to stop mirroring the arch form 760 for the upper and lower teeth 750 and 740. Therefore, adjustments made to the lower teeth 740 by moving the points 762A-N along the arch form 760 for the lower teeth 740 may not be replicated by the computer system for the upper teeth 750.

In some implementations, although the same arch form 760 can be used for the upper and lower teeth 750 and 740, the user can change a placement of the entire arch form 760 for one or both of the upper and lower teeth 750 and 740. For example, the computer system can define the arch form 760 for the lower teeth 740. The computer system can then replicate or mirror the arch form 760 for the upper teeth 750 and present the upper teeth 750 with the arch form 760 in a GUI at the user's device. The user may decide that the arch form 760 is closer to a lingual surface of some of the upper teeth 750 rather than a buccal surface of the upper teeth 750. Therefore, the user may select the arch form 760 for the upper teeth 750 and move the arch form 760 to better align with the buccal surface of the upper teeth 750. The computer system may not make this adjustment to the lower teeth 740, especially if the computer system had originally defined the arch form 760 for the lower teeth 740 in alignment with a buccal surface of the lower teeth 740.

FIG. 8 illustrates adjusting teeth 802A-N in a digital denture model to resolve IP contacts. The teeth 802A-N can be part of a lower arch 800, as shown in an occlusal view in FIG. 8. The computer system described herein can start by selecting a tooth at a midline 810 of the lower arch 800. The computer system can select the tooth 802A and work from the midline 810 to the last tooth 802N on one side of the lower arch 800. Once adjusting the teeth on the one side is complete, the computer system can return to the midline 810 and adjust the teeth back to a last tooth on the opposite side of the lower arch 800. The computer system can resolve any interference or collisions between the teeth 802A-N. The tooth 802A can be a central tooth having a contact point 808A with the adjacent tooth 802B. The computer system can identify a center point 804A and 804B, respectively, for each of the tooth 802A and 802B. The computer system can then define a vector 806A that goes through the center points 804A and 804B. The computer system can resolve interference of the teeth 802A and 802B by moving one or both of the teeth 802A and 802B along the vector 806A. The same operations can be performed to identify a contact point 808B between the tooth 802B and the tooth 802C, identify respective center points 804B and 804C, and define a vector 806B along which to move one or both of the teeth 802B and 802C to resolve any interference. The same operations can be performed to identify a contact point 808N between the tooth 802D and the tooth 802N, identify respective center points 804D and 804N, and define a vector 806N along which to move one or both of the teeth 802D and 802D to resolve any interference.

In some implementations, as described further in reference to FIG. 12, the computer system can first identify all center points 804A-N and all contact points 808A-N. The computer system can then define all vectors 806A-N. Then, the computer system can work tooth by tooth, from the midline 810 to the last tooth 802N on one side of the lower arch 800 to move the teeth 802A-N along the vectors 806A-N until the teeth 802A-N are in appropriate contact at the respective contact points 808A-N.

FIGS. 9A and 9B illustrate example movements for socking, vertically positioning, and/or correcting overbite in a digital denture model. FIG. 9A illustrates movements for socking, vertically positioning, and/or correcting overbite for anterior teeth. FIG. 9B illustrates movements for socking, vertically positioning, and/or correcting overbite for posterior teeth.

Referring to FIG. 9A, the computer system described herein can sock an anterior tooth 902, which is shown from a facial view 900 and a side view 910. A center point 908B can be identified for the anterior tooth 902, which can be used as a reference for socking, vertically positioning, and/or adjusting the overbite in reference to the tooth 902. To identify the center point 908B, the computer system can identify adjacent teeth 906A and 906B. The computer system can, for example, measure a total distance/width across the teeth 906A, 902, and 906B, and then find a midpoint of that total distance/width. The midpoint can be identified as the center point 908B for the tooth 902.

The computer system can also identify center points 908A and 908N for the adjacent teeth 906A and 906B, respectively. The computer system can define a buccal vector A as perpendicular to a reference line 909 that passes through the center points 908A, 908B, and 908N. The vector A can be used to adjust the tooth 902 buccal-lingually when socking the tooth 902. Furthermore, the computer system can identify a vector B, which can be used to adjust the tooth 902 up and down relative a lower tooth 904. The computer system can iteratively move the tooth 902 along the vectors A and B, as described further in reference to FIG. 13, until the tooth 902 is a predetermined or threshold distance D1 from the lower tooth 904 and/or the tooth 902 comes into contact with the lower tooth 904 at a desired or predetermined contact point 912 (which means the tooth 902 has achieved a desired overbite). The techniques described herein for socking the upper anterior tooth 902 can be performed by the computer system 102 on a tooth-by-tooth basis to adjust each anterior tooth in a digital denture model.

Referring to FIG. 9B, the computer system described herein can sock a posterior tooth 922, which is shown from a side view 920. A center point for the posterior tooth 922 can be determined by the computer system and as described in reference to at least FIGS. 9A and 13.

As described in reference to FIG. 9A, the computer system can iteratively move the tooth 922 from a starting position 926 to a socked position 928 along the vectors A and B. The computer system can iteratively move the tooth 922 until the tooth 922 is a predetermined or threshold distance D2 from a lower tooth 924 and/or the tooth 922 comes into contact with the lower tooth 924 at a desired or predetermined contact point 930 (which means the tooth 922 has achieved a desired overbite). For example, the computer system can iteratively move the tooth 922 in and out (e.g., buccal-lingually) along the vector A until a greatest or predetermined distance D2 is achieved. The computer system can also move the tooth 922 down along the vector B until the tooth 922 comes into contact with the tooth 924. The computer system can iteratively perform such movements and determine which of the movements along the vector A or the vector B result in a greatest vertical distance and thus a best socking-in fit between the upper posterior tooth 922 and the lower tooth 924. The techniques described herein for socking the upper posterior tooth 922 can be performed by the computer system 102 on a tooth-by-tooth basis to adjust each posterior tooth in a digital denture model.

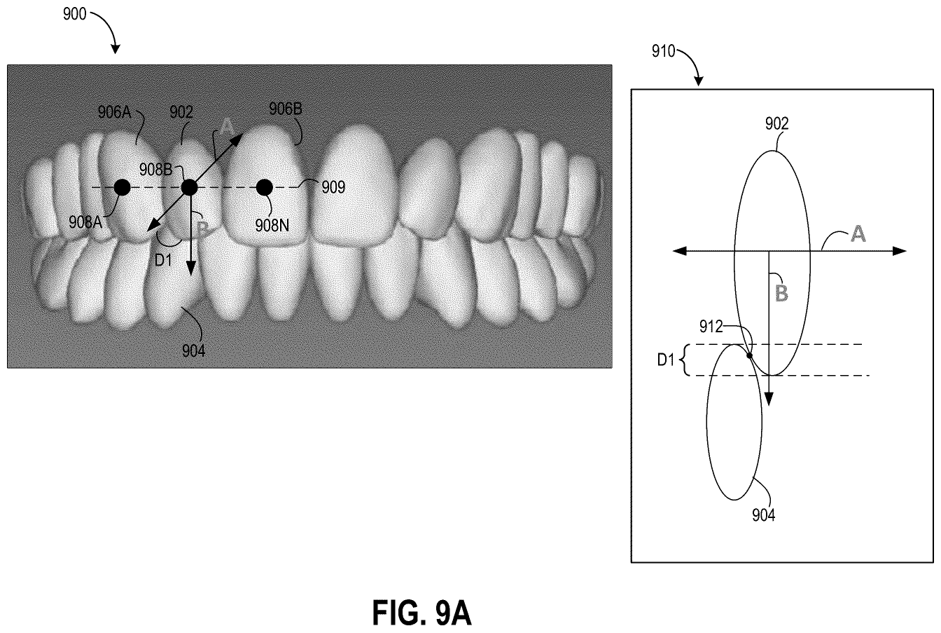

FIG. 10 is a flowchart of a process 1000 for automatically leveling teeth in a digital denture model. The process 1000 can be performed by the digital denture design system 102 described in reference to at least FIG. 1. The process 1000 can also be performed by any other type of computer system, computing device, cloud-based system, and/or network of computing systems. For illustrative purposes, the process 1000 is described from the perspective of a computer system.

Referring to the process 1000, the computer system can retrieve a digital denture model with labeled datums on teeth in the model in block 1002. Refer to at least blocks 302-318 in the process 300 of FIGS. 3A, 3B, and 3C for further discussion.

The computer system can identify a plane defined by a plurality of datums on each anterior tooth in the digital denture model (block 1004). For example, as shown in FIG. 6B, the computer system can define the plane as passing through 2 incisor tip edges or datums.

The computer system can then tip each anterior tooth according to the plane at a pivot point to level a top of the anterior tooth with an occlusal plane (block 1006). The pivot point can be defined by the plane. The pivot point can be defined by at least one of the datums for the anterior tooth. In some implementations, the pivot point can be a midpoint or center point of the tooth and/or the plane that is defined by the datums of the tooth. The computer system can tip the anterior tooth until its tip, for example, is parallel with the occlusal plane. In some implementations, the anterior tooth can be tipped until the plane defined by the plurality of datums is parallel with the occlusal plane.

In block 1008, the computer system can select a posterior tooth in the digital denture model.

The computer system can identify a pivot point for the posterior tooth as a midpoint between 2 or more marginal ridge datums for the posterior tooth (block 1010).

In block 1012, the computer system can rotate the posterior tooth around a line perpendicular to the midpoint to level the posterior tooth with the occlusal plane. When looking down at the posterior tooth, the line can be defined as perpendicular to the midpoint. Rotating the tooth around that perpendicular line can allow for the 2 or more marginal ridges to remain at a same distance away from the occlusal plane.

Optionally, the computer system can torque the posterior tooth using buccal and/or distal cusp datums so that the posterior tooth cusp(s) is parallel to the occlusal plane (block 1014). Refer to at least FIGS. 6A and 6C for further discussion about torqueing the posterior tooth.

The computer system can tip the posterior tooth mesially and/or distally using the 2 marginal ridge datums for the posterior tooth in block 1016. Refer to at least FIG. 6A for further discussion.

The computer system can then determine whether there are more posterior teeth to level in block 1018. If there are more posterior teeth to level, the computer system can return to block 1008 and repeat blocks 1008-1016 until all the posterior teeth have been leveled. The computer system can perform blocks 1008-1016 for each posterior tooth on an individual tooth-by-tooth basis. Once all the posterior teeth have been leveled, the computer system can proceed to block 1020.

If there are no more posterior teeth to level in block 1018, the computer system can perform block 1020, in which the computer system returns the digital denture model having the leveled teeth. The digital denture model can then be used to perform additional auto-setup operations described herein, such as snapping the teeth to arch forms.

FIG. 11 is a flowchart of a process 1100 for automatically snapping teeth to an arch form of a digital denture model. Snapping can include translation and rotation of a tooth to the arch form in order to position the tooth on the arch form. Sometimes, translating and rotating the tooth can be defined as a transformation of the tooth. Snapping the teeth can include moving one or more teeth in and out, or buccal-lingually. Snapping the teeth can also include rotating one or more teeth so that a tangent line faces an arch form and/or the one or more teeth achieve a predetermined or desired relationship with the arch form. Snapping the teeth can therefore include moving the teeth relative to the arch form and positioning the teeth on or a certain distance away from the arch form to achieve a predetermined, desired distance from the arch form.

The process 1100 can be performed by the digital denture design system 102 described in reference to at least FIG. 1. The process 1100 can also be performed by any other type of computer system, computing device, cloud-based system, and/or network of computing systems. For illustrative purposes, the process 1100 is described from the perspective of a computer system.

Referring to the process 1100, the computer system can retrieve a digital denture model with an arch form in block 1102. Refer to at least blocks 302-318 in the process 300 of FIGS. 3A, 3B, and 3C for further discussion.

The computer system can select an upper arch in the digital denture model in block 1103. Although the process 1100 is described as snapping teeth of the upper arch before snapping teeth of a lower arch, the process 1100 can also be performed in which the teeth of the lower arch are snapped to the arch form before the teeth of the upper arch are snapped to the arch form.

In block 1104, and for each tooth from a midline in the upper arch to a last molar in the upper arch, the computer system can snap the tooth tangent to the arch form curve. For example, the computer system can snap central incisors to the midline and tangent to the arch form curve (block 1106). The computer system can snap lateral teeth (block 1108). The lateral teeth can be snapped to the arch form similarly to the central incisors. The computer system can snap canines so that cusp tips of the canines are positioned relative the tangent line on the arch form curve and/or a threshold amount outside of the arch form curve (block 1110). As a result, the canines can be arranged to provide a desired overbite. For any tooth, the computer system can identify closest points on the arch form, translate the tooth to the closest points on the arch form, then apply a rotation to the tooth. For posterior teeth, for example, the computer system can use marginal ridge points as references for snapping. For incisors, as another example, the computer system can use incisal edge points/datums as references for snapping. For canines, as yet another example, the computer system can use one or more points or datums as references for snapping. Using a cusp tip datum on a canine, the computer system may translate the canine to the arch form but may not adjust the canine by rotation. The computer system may use additional datums on the canine to rotate accordingly.

Sometimes, tooth coordinate systems can be used for snapping the respective tooth. For example, once datums are identified for a tooth, the computer system can construct a coordinate system for the tooth based on the identified datums. The coordinate system can then be used by the computer system to appropriately align/snap the tooth to the arch form. The tooth coordinate systems can be predetermined by the computer system and stored with respective tooth libraries in a data store, as described herein. When the tooth libraries are retrieved and used for designing the patient's dental appliances, such as dentures, the computer system can load the teeth coordinate systems into the model and use the coordinate systems to appropriately rotate or otherwise adjust each tooth. The coordinate system is especially beneficial for rotating canines since the canines each only have 1 datum. The 1 datum may not provide enough information for determining how to rotate the canine to the arch form.

The computer system can, for each molar and upper arch bicuspid tooth, rotate the tooth so that respective marginal ridge datums are tangent to the arch form curve and position the tooth in and out so that the marginal ridge datums are aligned on the arch form curve (block 1112). For example, with a frame of reference of an occlusal view down on the arch form, the computer system can identify 2 marginal ridge datums on a molar and define a 2D line passing through those datums. The 2D line can be tangent to a spline curve of the arch form. A midpoint along the 2D line can be used for rotating the molar until the marginal ridge datums are tangent to the curve, then the molar can be moved in and out such that the marginal ridge datums are parallel to the spline curve and aligned with the curve.

Blocks 1104-1112 can be repeated for an opposite side of the arch form curve.

In block 1114, the computer system can select a lower arch in the digital denture model.

The computer system can, for each tooth from the midline to a last molar in the lower arch, snap the tooth tangent to the arch form curve (block 1116). For example, the computer system can snap lower incisors to a midline and inside the tangent line so that the arch form curve touches a buccal side of the lower incisors (block 1118). Whereas the computer system may move the upper incisors such that the tangent line is on an inside or lingual side of the upper incisors, the computer system can move the lower incisors such that the tangent line id touching a facial or buccal side of the lower incisors. As a result, the upper teeth are arranged to be slightly in front of the lower teeth, which allows for a desired overbite for the patient.

The computer system can snap canines so that cusp tips of the canines are positioned relative the tangent line inside the arch form curve or a threshold distance inside of the arch form curve (block 1120). As mentioned above, the upper canines can be moved by the computer system relative the tangent line outside the arch form curve.

The computer system can, for each molar and lower posterior tooth, translate the tooth lingually so that a respective buccal cusp tip is positioned on the arch form curve (block 1122). The lower arch teeth can be rotated in blocks 1116-1122 similarly to the upper arch teeth rotations described in reference to blocks 1104-1112 so that a reference line going through marginal ridge datums in each lower tooth is tangent to the curve. In other words, the computer system can move each posterior tooth so that a central groove/trough of the tooth is aligned with and on the spline curve (therefore, the corresponding marginal ridge datums are be aligned with and on the spline curve).

The computer system can return the digital denture model with snapped teeth in block 1124.

Optionally, the computer system can automatically adjust the snapped teeth to resolve collisions and/or interference (block 1126). Refer to at least FIGS. 8 and 12 for further discussion.

FIG. 12 is a flowchart of a process 1200 for automatically resolving IP contacts in a digital denture model. The process 1200 is further described in reference to FIG. 8. The process 1200 can be performed by the digital denture design system 102 described in reference to at least FIG. 1. The process 1200 can also be performed by any other type of computer system, computing device, cloud-based system, and/or network of computing systems. For illustrative purposes, the process 1200 is described from the perspective of a computer system.

Referring to the process 1200 in FIG. 12, the computer system can retrieve a digital denture model with an arch form in block 1202. Refer to at least blocks 302-328 in the process 300 of FIGS. 3A, 3B, and 3C for further discussion.

The computer system can generate a bounding box around each tooth in the digital denture model (block 1204).

The computer system can, for each tooth, identify a center point of the tooth as a center point in the respective bounding box (block 1206). The center point of the tooth can be an absolute center of a volume of the tooth.

The computer system can identify vectors between the center points of the teeth in block 1208. The vectors can define directions to move the adjacent teeth in order to achieve a desired IP contact between the adjacent teeth. The vectors for all the teeth can be identified and locked in before adjusting the teeth so that once the computer system moves the teeth along the vectors, a corresponding length of each vector does not change as well. As a result, relative orientation can be maintained between the adjacent teeth while removing any potential interference, overlap, and/or collisions between the adjacent teeth.

In block 1210, the computer system can select a first tooth that is in a defined position. The defined position can be near a first side of a midline of the arch form. Thus, the computer system can select the first tooth that is positioned at the midline of the arch form. The computer system can then work along one side of the arch form starting from the first tooth to a last tooth on the side of the arch form. As described further below, once the computer system completes adjusting the teeth on the one side of the arch form, the computer system can select teeth on another side of the arch form that is opposite the first tooth at the midline.

The computer system can, in block 1212, identify an nth tooth that is adjacent the first tooth. For example, the computer system can identify a second tooth that is to a right or left side of the first tooth.

Then, the computer system can move the first and/or nth tooth along the vector between the first and the nth tooth to maintain relative orientation, remove teeth overlap, and/or put the first tooth in contact with the nth tooth at a predetermined or desired contact point (block 1214). The center points of the first and nth teeth can be used to maintain relative orientation of the teeth during subsequent passes through the auto-setup process. For example, the computer system can perform an initial pass through of resolving IP contacts between adjacent teeth. A relevant user can then modify one or more of the teeth (e.g., rotate, tip, and/or torque some teeth). The user modifications can be locked in for the modified teeth so that a next time that the computer system passes through the auto-setup operations, the user modifications are not undone and the center points are used for those subsequent auto-setup passes.

The computer system can determine whether there are still adjacent teeth in block 1216. For example, the computer system can determine whether another tooth is adjacent the nth tooth, the another tooth being different than the first tooth. If there is still at least one adjacent tooth, the computer system can adjust the vector between the nth tooth and the next adjacent tooth in block 1218, and then move the nth tooth along the adjusted vector relative to the next adjacent tooth as described in block 1214.

If there are no more teeth adjacent the nth tooth, then the computer system can determine whether there is still another side of the midline that is opposite the defined position for which to resolve IP contacts (block 1220). If there is another side opposite the midline to assess, the computer system can select a first tooth near the side opposite the midline in block 1222 and repeat blocks 1212-1214 until there are no more adjacent teeth to assess on the side opposite the midline.

If the side opposite the midline has been adjusted in block 1220, the computer system can return the adjusted model in block 1224. The adjusted model, as described herein, can be presented in a GUI at the user's device. The adjusted model can also be used by the computer system to perform one or more other auto-setup operations described herein, such as adjusting vertical positioning of each tooth in the model.

FIG. 13 is a flowchart of a process 1300 for automatically socking upper teeth in a digital denture model. The process 1300 can be performed to sock upper anterior teeth and upper posterior teeth. In some implementations, the process 1300 can first be performed to sock the upper anterior teeth. The process 1300 can then be performed a second time to sock the upper posterior teeth. The process 1300 is further described in reference to FIGS. 9A and 9B.

The process 1300 can be performed by the digital denture design system 102 described in reference to at least FIG. 1. The process 1300 can also be performed by any other type of computer system, computing device, cloud-based system, and/or network of computing systems. For illustrative purposes, the process 1300 is described from the perspective of a computer system.

Referring to the process 1300 in FIG. 13, the computer system can retrieve a digital denture model with an occlusal plane in block 1302. Refer to at least blocks 302-330 in the process 300 of FIGS. 3A, 3B, and 3C for further discussion.

The computer system can select an upper arch of teeth in the digital denture model in block 1304.

In block 1306, the computer system can identify a center point between 3 teeth in the upper arch to define a buccal vector as perpendicular to the center point. The buccal vector can be defined for a tooth between 2 of the 3 teeth. The computer system can perform block 1306 for each tooth so that a buccal vector can be defined per tooth. The buccal vectors for the teeth can be defined before proceeding to the next blocks in the process 1300, in some implementations. For a last molar, the computer system can use a second-to-last buccal vector that was defined for the upper arch as the last molar's buccal vector.

The computer system can, in block 1308 and for each upper tooth, iteratively adjust the tooth bucally, lingually, down, and/or up based on the buccal vector. For posterior teeth, for example, the computer system can iteratively move the tooth down vertically until it comes into contact with a lower arch tooth. For anterior teeth, these iterative adjustments can be performed in order to correct overbite of the anterior teeth.

The computer system can measure a distance between the adjusted upper tooth and a lower arch tooth in block 1310.

In block 1312, the computer system can determine whether the distance between the adjusted upper tooth and the lower arch tooth is within a predetermined threshold distance. The predetermined threshold distance can be different based on whether the adjusted upper tooth is a posterior tooth or an anterior tooth. The predetermined threshold distance can also be different based on a particular type of tooth that is being adjusted. For example, the predetermined threshold distance for central teeth and/or canines can be approximately 2 mm. As another example, the predetermined threshold distance for lateral teeth can be approximately 1.5 mm. One or more other threshold distances can be provided/adjusted by a relevant user and used in block 1312.

If the distance is within the predetermined threshold distance, the computer system can proceed to block 1318 in which the computer system can determine whether there is another tooth in the upper arch form to adjust. If there is another tooth to adjust, the computer system can return to block 1306 and repeat blocks 1306-1312 until all teeth in the upper arch form have been adjusted.

If there are no other teeth in the upper arch to adjust, the computer system can return the digital denture model with the adjusted teeth in block 1320. As described herein, the digital denture model can be outputted in a GUI at a user device. The digital denture model can also be used by the computer system to perform one or more other auto-setup operations described herein, such as iterating through one or more position adjustments of one or more teeth until a desired denture design criteria is achieved.

Referring back to block 1312, if the distance between the adjusted upper tooth and the lower arch tooth is not within the predetermined threshold distance, then the computer system can reduce the distance in half (block 1314) and adjust the upper tooth in an opposite direction of the previous adjustment(s) by the reduced distance amount (block 316). The computer system can then return to block 1310 and iterate through blocks 1310-1316 until the distance between the adjusted upper tooth and the lower arch tooth is within the predetermined threshold distance.

As an illustrative example, the computer system can implement a binary algorithm in which the computer system moves a central tooth in, out, and down to contact a lower arch tooth with a distance of 2 mm (millimeters) from the lower arch tooth. If the movement down is overshot, then the computer system can reduce the distance moved in half and move the central tooth back up, in, and/or out in an opposite direction by the half distance. The computer system can measure the distance again after this second adjustment. If the central tooth has been moved up too high, then the computer system can cut that distance in half and move the central tooth back down by the halved distance. On the other hand, if the central tooth has not been moved enough, then the computer system can move the tooth out by the halved distance (or any other amount of distance). The computer system can therefore iterate through this process until the desired distance is achieved between the central tooth and the lower arch tooth.

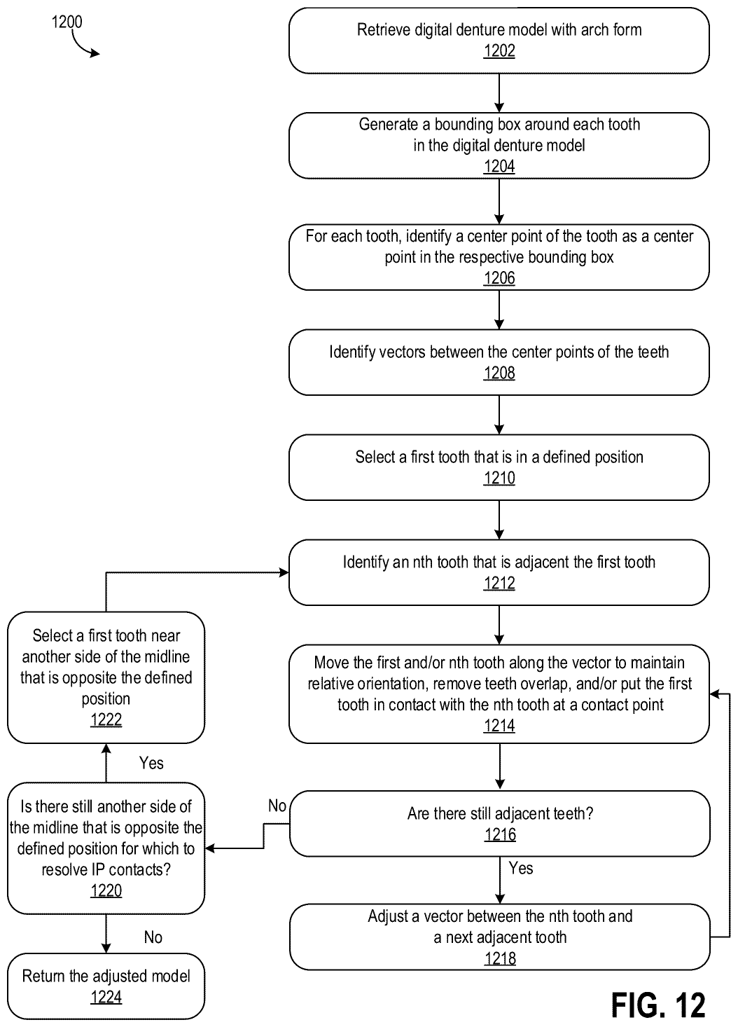

FIGS. 14A and 14B illustrate example GUIs 1400 and 1402, respectively, that may be generated during an automated process of positioning selected tooth library teeth in a digital denture model and automatically aligning and leveling each tooth of the selected tooth library. FIG. 14A shows a digital denture model 1405 and a selected tooth model 1415. The selected tooth model 1415 is automatically positioned by the computer system described herein on the digital denture scan 1405 to align each tooth with a determined arch and to position each tooth so that it is in a position that most closely matches teeth of a digital denture. In some implementations, an ICP algorithm can be used by the computer system to determine a best fit. The application of the ICP algorithm can be displayed in the GUI 1400 to illustrate to a technician positioning of the teeth and degree of difference that may remain.

In some implementations, an overlay of the library tooth mold tooth and the digital model from the denture scan can be displayed to the user in the GUI 1400 with a color map or heat map to illustrate differences between the library tooth mold tooth and the existing scan. In some implementations, the colors of the color map indicate varying degrees of difference between the library tooth mold and the existing scan. For example, green can indicate that the library tooth mold and the existing scan are very close, yellow may indicate that the positioning can be improved, and red can indicate that there is a large degree of difference between the library tooth mold and the existing scan.

In FIG. 14B, the teeth of the selected tooth model 1405 are positioned in the digital denture model 1415 in a best-fit position 1425, and differences between the teeth of the digital denture model 1415 and the teeth of the selected tooth model 1405 are illustrated to a user by a heat map or color map. This information may be beneficial to identify wear patterns and areas of the denture library teeth that may need to be modified in the replacement denture.

In FIG. 14B, areas where the teeth of the selected tooth model 1405 extend beyond boundaries of the teeth of the digital denture model 1415 can be colored green (for example position 1435). The technician can use such a display to make further adjustments to the digital denture model teeth. In some implementations, a technician or dentist can use the GUI 1402 to understand changes in the tooth shape from the patient's chewing or grinding habits, and can make recommendations to the patient based on the information.

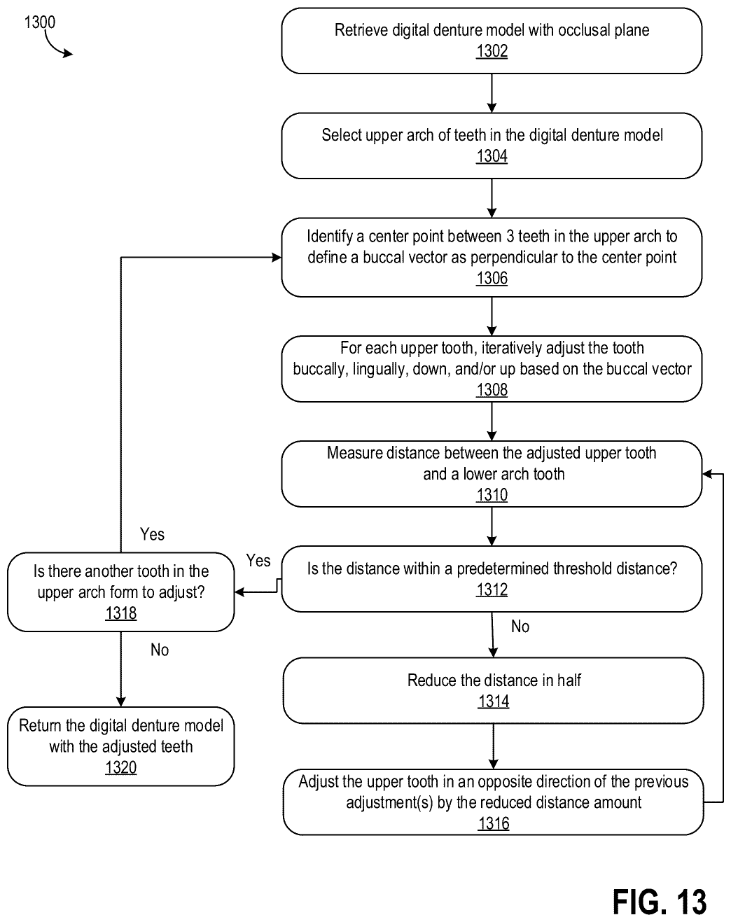

FIGS. 15A, 15B, and 15C illustrate example GUIs 1500, 1502, and 1504, respectively, that may be generated during a process of automatically aligning and leveling each tooth of a selected tooth library in a digital denture model. For example, FIG. 15A shows the selected library tooth mold 1505 in an initial position with regard to the digital denture model 1515 in the GUI 1500. Each tooth from the selected library tooth mold 1505 is then fit to the corresponding tooth of the digital denture model 1515 by aligning the tooth with the arch of the digital denture model 1515 and leveling the tooth with respect to the occlusal plane, as described herein.

FIG. 15B shows, in the GUI 1502, a partial completion of the process of FIG. 15A, where teeth on the left side of the digital denture model 1515 have been leveled and aligned to a best-fit position 1525, and teeth of the selected library tooth mold 1505 on a right side of the digital denture model 1515 are not yet fit by the algorithm.

FIG. 15C shows, in the GUI 1504, the completed process of FIG. 15A, where all teeth are in a best-fit position 1525, and areas that the teeth of the selected tooth model 1505 extend beyond the boundaries of the teeth of the digital denture model 1515 are colored according to a heat map for presentation to the technician (for example position 1535).

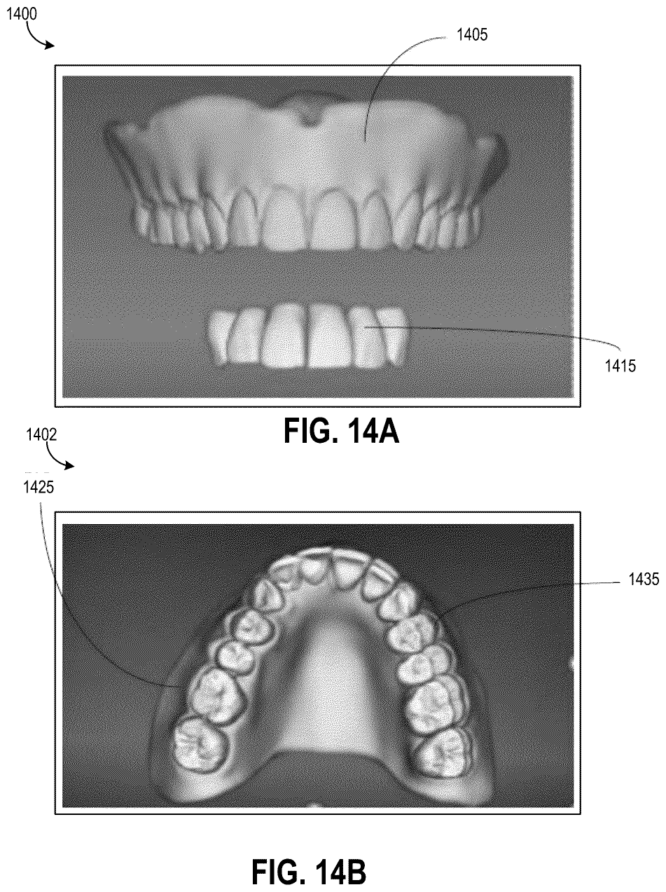

FIG. 16 illustrates an example GUI 1600 that may be generated during a process of manually adjusting a position of a selected tooth of a tooth library in a digital denture model. The GUI 1600 can receive user input to reposition a digital denture tooth. In this example, a user has selected one of the digital denture teeth, an upper bicuspid 1602. The selected tooth 1602 can be highlighted or otherwise visually presented in an indicia that is different than the visual presentation of the other teeth. Here, for example, the selected tooth 1602 is presented in a light blue indicia. A user may provide user input to cause the selected digital denture tooth 1602 to move in a mesial and/or a distal direction. In this example, the computer system can detect contact with digital denture teeth that are adjacent the selected tooth 1602 to automatically prevent further movement of the selected digital denture tooth 1602.

The user may, for example, select the digital denture tooth 1602 by using a mouse to click on the representation of the digital denture tooth in the GUI 1600. In some implementations, the user may select the digital denture tooth 1602 by touching a touchscreen at their respective computing device. The selected digital denture tooth 1602 may be displayed using different coloring or shading than the other digital denture teeth, as mentioned above. The GUI 1600 may also receive a user input to move the selected digital denture tooth 1602. In some implementations, the user input can be a drag input such as a click-and-drag or touch-and-drag. Based on the direction of the drag, the computer system may move the digital denture tooth 1602 in a corresponding direction. In some implementations, the movement may be in a direction that is parallel to an occlusal plane.

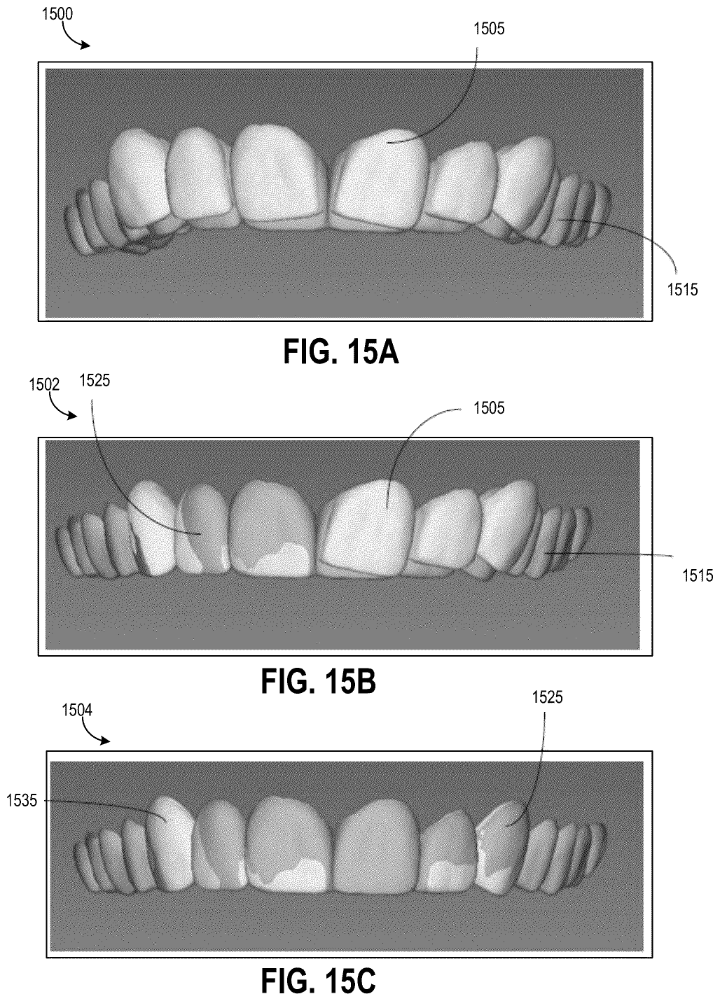

FIG. 17 illustrates an example GUI 1700 that may be generated during a process of manually adjusting a position of a selected group of teeth 1702 of a tooth library in a digital denture model. In this example, three digital denture teeth are selected in the group 1702 and are being repositioned (e.g., manually via user input from a user). As the digital denture teeth in the group 1702 can be moved, by the computer system described herein, in response to user input. Each tooth in the group 1702 can be individually moved into contact with the opposing dentition/teeth. For example, the three selected digital denture teeth in the group 1702 may be moved in a distal direction via the user input. As the computer system moves the group 1702 of teeth according to the user input, each tooth in the group 1702 can move in a gingival or occlusal direction to maintain occlusal contact with opposing dentition while avoiding interference (e.g., overlap or collision of the digital denture teeth). These tools may be used to rotate and reposition the upper digital denture teeth into a bilaterally balanced, lingualized occlusion (e.g., by rotating the upper teeth so that the buccal cusps are oriented further in the buccal direction).

FIGS. 18A and 18B illustrate example GUIs 1800 and 1802, respectively, including toolboxes 1804 and 1806 with user-selectable options that may be used while designing and/or adjusting a digital denture model. The toolboxes boxes 1804 and 1806 can be used and presented to guide a user and allow the user to manipulate and position teeth and soft tissue in creation of the digital denture model.

FIG. 18A, for example, illustrates the GUI 1800 with the dialog boxes 1804 and 1806 that can be generated by the denture design system described herein to receive user input to reposition a digital denture tooth. In this example, a user has selected one of the digital denture teeth 1808 (an upper molar). The GUI 1800 is configured to allow the user to rotate the selected digital denture tooth 1808 by providing user input. Here, the digital denture tooth 1808 is being rotated by the computer system described herein and based on the user input about an axis that is represented on the GUI 1800 by a sphere 1810. As the digital denture tooth rotates, it is automatically moved in an occlusal or gingival direction to maintain contact and avoid overlap with opposing dentition.

In some implementations, the GUI 1800 may allow the user to iterate through the techniques described herein for positioning digital denture teeth repeatedly and in any order. In some implementations, the technician can make adjustments to teeth on one side of a midline and can then implement same or similar adjustments to teeth on the other side of the midline (e.g., with one click and/or selection of a selectable option/control/button in one of the toolboxes 1804 and 1806). In some implementations, the technician can select teeth by clicking on each tooth, by selecting teeth using a checkbox or button in the GUI 1800, and/or by another mechanism described herein.

FIG. 18B illustrates an example GUI 1802 with the toolboxes 1804 and 1806 to allow the user to select, position, choose, and manipulate digital denture teeth and soft tissue to prepare a digital denture model. As shown in both FIGS. 18A and 18B, the first toolbox (“Denture Design toolbox”) 1804 can include buttons for user-selection of various actions that the user can select to automate the denture model design. The actions include, but are not limited to, the ability to clean, orient, level, fix bite, fit arches, choose denture teeth, fit denture teeth, and create base functions. The second toolbox (“Setup Toolbox”) 1806 includes buttons for selection of various actions that allow the user to manipulate or change the automated suggestions generated by the first toolbox 1804. The actions include, but are not limited to, an ability to view data about each tooth (“tooth datums”), manipulate the arch form, set the occlusion of the teeth, move the teeth to occlusion, open an auto-setup toolbox, and pick individual or groups of teeth for moving. The individual or groups of teeth can then be positioned, forced into contact, torqued, leveled, aligned, and animated based on the user interaction with the second toolbox 1806.

In the first toolbox 1804, the clean function can be performed by the computer to process the digital denture model to remove or fix irregularities, such as holes, metallic inclusions, or features that are not part of the dentures that are included in the model due to artefacts of the scan to produce the denture model. The orient function can be performed by the computer system to automatically orient the digital denture scan in the user interface, and/or can allow the user to rotate, pivot, or otherwise move the digital denture model in three-dimensional space to inspect or orient the model. In some implementations, the orient button can also cause the computer system to return the model to a neutral position. The level button can be performed by the computer system to automatically level the denture model as if on a flat surface. In some implementations, the level button can cause the computer system to automatically level one or both of the soft tissue and the upper or lower denture teeth to a horizontal surface.

The fit bite and fit arches buttons can allow the user to interact with the digital denture model to cause the computer system to automatically process the model to suggest an appropriate bite and arch to fit the model, and to adjust the suggested bite and arch. For example, in some implementations, the computer system can suggest a best fit based on a variety of algorithms and measurements of the digital denture model, and the user can then use the second toolbox to further manipulate the arch form and occlusion position of the teeth using the “arch form,” “set occ,” and “move to occ” buttons. The user may select points on the suggested arch or points on the upper and lower dentures and move the points to adjust the suggested best fit.

The user can use the second toolbox 1806 to adjust best fit models produced using the first toolbox 1804 to incorporate additional information that is not available to the computer system, such as patient preference, adjusting the positioning of the teeth to accommodate a dental issue, and/or attempting to recreate a unique aspect of the patient's own teeth or smile.

The choose denture teeth button on the first toolbox 1804 can allow the user to select an initial set of denture teeth from multiple tooth libraries. As described above, the user can select from a suggested set of best fit tooth libraries. The user can also swap out a selected set of teeth for another, or choose particular teeth from another library using the choose denture teeth button. The fit denture teeth can also suggest, by the computer system, an initial position of each of the teeth from the tooth library, for example using an ICP algorithm to position the teeth. A base can be automatically created, by the computer system, using the “create base” button of the first toolbox 1804. Using the create base button, the computer system can automatically suggest a base formed to the soft tissue of the patient from the scan data.

Using the second toolbox 1806, the user can select individual teeth or groups of teeth for manipulation and positioning in the digital denture model. Once the user has selected one or more teeth, the user can adjust the teeth by interacting with the position, contact, torque, level and align buttons. The user can also animate the digital denture model to view the model from different perspectives, view the upper and lower denture models moving in relation to each other, or viewing the animation of the adjustments made to the teeth in the program to review the model. The buttons and actions available in the first and second toolboxes 1804 and 1806 are illustrative only, and additional buttons can be added to provide more features and actions that the user can take to design, review, and transmit denture designs for manufacture. Though the buttons are shown in two toolboxes 1804 and 1806, the buttons can be presented to the user in a single toolbox, or more than two toolboxes. In some implementations, the actions can be accessible through use of a drop down menu, voice controls, joy sticks, radio buttons, or any other suitable mechanism for selection. By automatically producing suggested fits, positions and designs, the computer system can advantageously and dependably produce high quality denture designs. Allowing the user (for example, a technician) to make adjustments to the design enables the designs to be further personalized based on patient preferences, feedback, and needs that are not accounted for in the automatic design program.

The GUIs 1800 and 1802 can optionally be displayed with one or more additional or other dialog boxes, toolboxes, and/or selectable options. For example, A dialog box with selectable controls can be presented for editing one or more teeth and/or a digital model, more generally. The selectable controls can include, but are not limited to, picking/selecting one or more teeth, using a wand over or more teeth (which allows the user to draw a free-form shape in the respective GUI to select vertices of a mesh for the digital model inside the shape), using paint or changing a color of one or more teeth, flooding one or more teeth (which allows the user to click on and select parts of the GUI that are unselected but within selected parts of the GUI), shrinking one or more teeth, expanding one or more teeth, pushing one or more teeth, pulling one or more teeth, smoothing one or more teeth, filling holes or other gaps in the digital denture model, sculpting the model and/or one or more teeth, identifying and/or placing one or more datums for one or more teeth, translating one or more datums (e.g., performing a translation of action(s) from 1 datum to another datum), translating one or more vectors, transforming one or more steps (which allows the user to control incremental amounts of movement in mesial, distal, vertical, etc. directions for any tooth in the model in terms of degrees and/or distances), performing one or more mouse transforms (which allows the user to click and drag on something in the respective GUIs to perform actions/movements such as tooth rotations and translations), copying one or more actions/edits, deleting one or more edits, and/or undoing one or more edits.

FIG. 18C illustrates example GUIs 1810 and 1820 with respective toolboxes 1812 and 1822 of selectable options for automatically designing and/or adjusting lower and upper teeth, respectively, in a digital denture model. The GUI 1810 includes the toolbox 1812 with selectable options for auto-adjusting lower teeth. The GUI 1820 includes the toolbox 1822 with selectable options for auto-adjusting upper teeth. As shown in the GUIs 1810 and 1820, some options in the toolboxes 1812 and 1822 can be automatically selected (e.g., preset, preselected) and performed by the computer system as part of an auto-setup process as described herein. A relevant user, such as a technician, can select and/or deselect one or more other options in either the toolboxes 1812 or 1822 to customize what operations are performed as part of the auto-setup process.

The computer system described herein can perform one or more similar, same, and/or different operations to auto-setup/auto-design upper teeth and lower teeth. For example, to auto-setup the lower teeth, as shown by the toolbox 1812 in the GUI 1810, the computer system can perform one or more of the following operations: level molar buccal-lingual cusps, level molar marginal ridges, level bicuspid marginal ridges, level incisors, snap to arch form, adjust IP contacts, anchor molars, and/or snap to occlusal plane. Any of these operations can be default-selected and performed by the computer system, unless the relevant user provides input to select or deselect one or more of these operations.

To auto-setup the lower teeth, as shown by the toolbox 1822 in the GUI 1820, the computer system can perform one or more of the following operations: level molar buccal-lingual cusps, level molar marginal ridges, level bicuspid marginal ridges, level incisors, snap to arch form, adjust IP contacts, anchor molars, snap to occlusal contact, adjust overbite, and/or nest posteriors. Snapping to occlusal contact can cause the computer system to automatically push the upper teeth into occlusion contact with the lower teeth. Adjusting overbite can cause the computer system to automatically adjust the overbite as described herein for upper anterior teeth. Nesting posteriors can cause the computer system to automatically sock in upper posterior teeth, as described herein.

The lower teeth can be auto-setup first, then the upper teeth can be auto-setup. In some implementations, the lower and upper teeth can be auto-setup at a same time. Sometimes, the upper teeth can be auto-setup first, followed by the lower teeth. The auto-setup of the lower teeth can also impact the auto-setup of the upper teeth. For example, an arch form and/or repositioning of lower teeth relative to the arch form can cause the computer system to move the upper teeth along the arch form and adjust an overbite of the upper teeth based on the positioning of the lower teeth relative to the arch form. Various other implementations are also possible.

FIGS. 19A, 19B, and 19C illustrate schematic diagrams of an example digital denture model and example denture teeth. The FIGS. 19A, 19B, and 19C illustrate aspects of fitting of the selected denture tooth library to the digital denture model. FIG. 19A displays a digital reference denture model 2102 along with a portion 2104 of a selected denture tooth library. In this example, the portion 2104 includes denture tooth 2106a, denture tooth 2106b, denture tooth 2106c, denture tooth 2106d, denture tooth 2106e, and denture tooth 2106f. The portion 2104 may be displayed for a selected denture tooth library. A user may review and approve selection of the denture tooth library using a user-actuatable control (not shown) displayed on the user interface 2100. In some implementations, the user interface 2100 may include one or more additional user-actuatable controls to load or scroll through different denture tooth libraries.

The denture teeth models may be aligned using, for example, an iterative alignment process, such as an iterative closest point (ICP) alignment performed by the computer system described herein. Iterative closest point alignment may be performed by iteratively (e.g., repeatedly) associating selected points (e.g., vertices) from the denture tooth model with the closest points from the identified portion of the digital reference denture model, estimating a transformation (e.g., a rotation and translation) of the denture tooth model to more closely align the selected points from the denture tooth model to the associated closest points from the portion of the digital reference denture model, and applying the transformation to the denture tooth model. In some implementations, the selected points on the denture tooth model are on an anterior surface of the denture tooth model. The selected points may be identified in advance and stored with the denture tooth model (e.g., as labels associated with specific vertices).

The alignment process may continue for a specific number of iterations or until the transformation calculated/applied by the computer system during an iteration is below a specific threshold. The aligned denture tooth may be compared to the portion of the denture scan to calculate a similarity value. In some implementations, portions of the denture tooth model are weighted differently when computing a similarity score. For example, the incisal edge may be assigned a lower weight than the labial surface. This weighting may compensate for the fact that the incisal edges of the teeth in the digital reference denture model are more likely to be damaged or worn down to long-term use.

After the first denture tooth model is aligned to the digital reference denture model, an adjacent tooth may be positioned next to it by the computer system. After the adjacent denture tooth model is initially positioned next to the aligned denture tooth model, the adjacent denture tooth model may then be aligned with the digital reference denture model (e.g., using an alignment technique such as iterative closest point). This process may continue to be performed by the computer system, working from the anterior dentition back to the posterior dentition, one tooth at a time until all of the teeth have been aligned to the digital reference denture model.

Multiple denture tooth models from different libraries may be aligned and compared by the computer system. The denture tooth library containing the most similar denture tooth model (e.g., based on the calculated similarity values) may be selected. In some implementations, denture tooth models from a subset of the different libraries can be used by the computer system. An initial filter (or selection) process may be used to reduce a number of different libraries that are considered. The initial filter process may be based on a width value of one or more anterior teeth. The initial filter process may be based on biographic information corresponding to the patient.

The initial filter process may also be performed by the computer system and based on extracting a shape from the selected portion of the digital reference denture model. For example, multiple horizontal slices of the portion may be generated by the computer system (e.g., by computing the intersection of a horizontal plane with the portion) and compared to each other to determine a general shape of the tooth. For example, this process may be performed by the computer system to determine that the portion of the digital reference denture model has teeth with a square, ovoid, or tapering shape. A subset of denture tooth libraries may then be identified based on the determined shape. This subset may be aligned and compared to the portion of the digital reference denture model to calculate a similarity value.

The initial filter process may also be based on other properties of the reference denture model that are manually or automatically determined. For example, one or more of a point angle, line angle, or labial convexity value may be determined for an anterior tooth portion of the reference denture model. As shown in FIGS. 19B and 19C, the denture tooth 2106c is shown with an indicator L of a line angle, an indicator P of a point angle, and indictor C of labial convexity. The indicator L of the line angle shows an angle value that may be determined for the library tooth by determining the angle of the distal edge of a tooth. The indicator P of the point angle shows a value that corresponds to the roundness of an incisal corner of the tooth. The indicator C shows a value that corresponds to the convexity of the labial surface of the tooth. In some implementations, more than one point angle, line angle, or labial convexity value is determined for the patient's dentition as the tooth may be asymmetric. These and other properties of a patient's teeth may be determined and used to guide the selection of a denture tooth library in at least some implementations.

As described above, when dentures for both the upper and lower dental arches are being produced, a single library of denture teeth may be selected and used for both the upper and lower dental arches in some implementations. In other implementations, separate libraries of denture teeth are selected for the upper dental arch and the lower dental arch. Further, different libraries or different variants of library teeth may be selected. For example, different libraries or variants within a library may be selected for antimeres so as to provide asymmetry that may create a more natural appearance for the denture.

FIG. 20A illustrates example libraries of digital denture teeth. FIG. 20B illustrates differences between the digital denture teeth of FIG. 20A with an overlay color map. More specifically, FIG. 20A shows a first set of teeth from a tooth library 2003, a second set of teeth from a tooth library 2007, and a third set of teeth from a tooth library 2009. Although some dimensional differences might be detected upon visual inspection of the three sets of teeth 2003, 2007, 2009, the full dimensional differences between the models can be seen in FIG. 20B by overlaying the three sets in a color map 2011. These differences can have an outsized impact on patient acceptance of the dentures, as they impact the look, feel, and function of the dentures

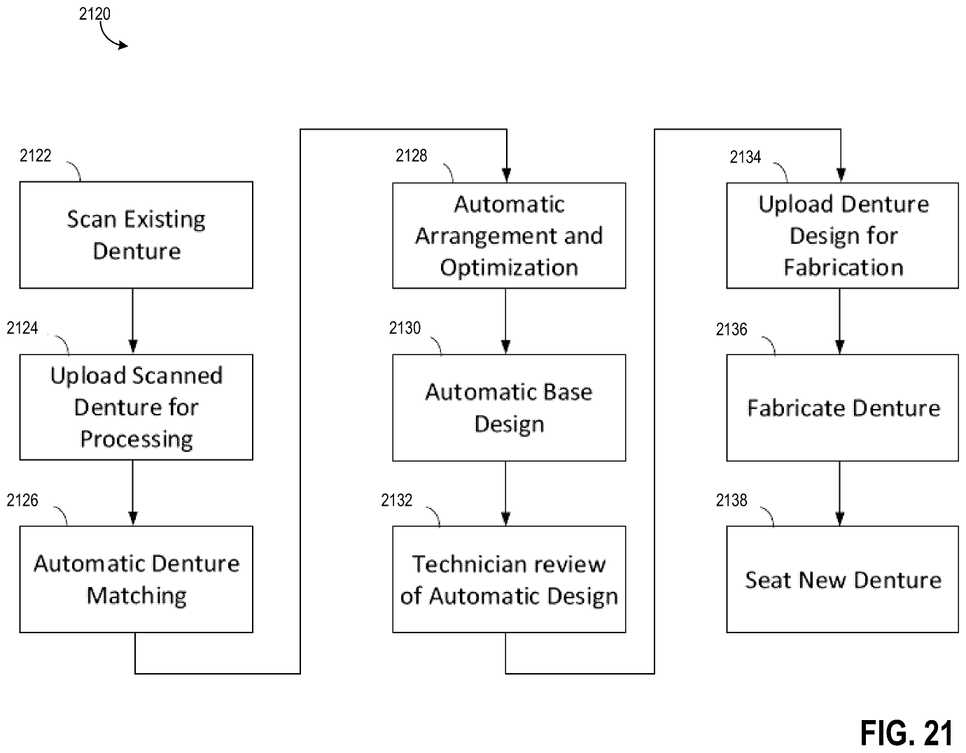

FIG. 21 is a flowchart of an example process 2120 for automatic design and fabrication of a replacement denture. The process 2120 can be performed by the digital denture design system 102 described in reference to at least FIG. 1. The process 2120 can also be performed by any other type of computer system, computing device, cloud-based system, and/or network of computing systems. For illustrative purposes, the process 2120 is described from the perspective of a computer system.

Referring to the process 2120 in FIG. 21, at block 2122, an existing denture is scanned. At block 2124, the scanned denture can be uploaded for processing or transferred to a computing device for local processing, such as the computer system described herein. At block 2126, a digital denture model based on the scanned denture can be automatically matched to a digital tooth library that most closely matches the teeth of the existing denture. At block 2128, the teeth of the digital tooth library matched and selected at block 2126 can be automatically arranged and optimized to fit the digital denture model based on the scanned denture.

At block 2130, the base of the digital denture model can be automatically designed by the computer system. For example, the computer system can automatically design the base with a desired thickness (e.g., 2.5-3 mm thick) that also fits against an intaglio surface in the patient's mouth (e.g., soft tissue on an arch in the patient's mouth where there are no teeth). The computer system can automatically design sockets in the base that are defined to receive and appropriately fit the teeth of the designed denture model. The computer system can also automatically design margins in the sockets, which can be a gingival margin of a soft tissue bump next to a tooth.

At block 2132, a technician may review and approve the automated design. In some implementations, as discussed above, the technician can review and adjust the design after each step. In some implementations, the technician does not review, or is not required to review, until all automatic design processes have been completed by the computer system. In some implementations, the technician can make adjustments and review after each automatic processing step, and may also approve the automatic design prior to transmission of the design for fabrication.

At block 2134, the denture design can be uploaded for fabrication, or is otherwise transmitted to a fabrication facility. In some implementations, the fabrication facility is on-site at the dentist office. In some implementations, the fabrication facility is a separate entity from the dentist office. At block 2136, the denture can be fabricated based on the digital denture design produced by the automatic design process. At block 2138, the new denture can be received and seated by a dentist or technician for the patient.

The automatic design of the replacement denture can save cost and time and can also more efficiently produce high-quality replacement dentures that are similar to the original or existing dentures. Because the automatic design can implement algorithms to iteratively match teeth from tooth libraries to a digital denture model produced from a denture scan, the resulting denture model can be more similar to the original dentures than models produced by a technician relying on visual inspection of the dentures to match to a tooth library. Accordingly, there can be less variability between dentures produced by different technicians, and the produced dentures may be more likely to be accepted by the patients as being similar or substantially the same as previous dentures in function, fit, and aesthetics.

FIG. 22 is a conceptual diagram of system components for selecting tooth libraries. The computer system 102 described herein can include a tooth library selection engine 2210, an auto-design denture setup engine 2200, a perfect smile setup engine 2202, and/or an auto-design implant setup engine 2206. The computer system 102 can include additional or fewer engines. The tooth library selection engine 2210 can communicate (e.g., wired and/or wirelessly via networks described herein) with one or more of the engines 2200, 2202, and 2206.

The auto-design denture setup engine 2200 can transmit a request (block A, 150) to the tooth library selection engine 2210 for a subset of tooth libraries and/or at least one candidate tooth library to be used in designing dentures for a particular patient. Upon receiving the request, the tooth library selection engine 2210 can perform the techniques described herein to select the tooth library or libraries. For example, the engine 2210 can determine aggregated tooth measurements for the particular patient (e.g., aggregated or combined tooth widths) and then select the tooth library having respective aggregated tooth measurements that are within a threshold range of or closest to the aggregated tooth measurements for the particular patient. The engine 2210 can then transmit the selected tooth library or libraries to the auto-design denture setup engine 2200 (block B, 152). The engine 2200 can then auto-design the dentures for the particular patient using the selected tooth library or libraries as described throughout this disclosure.

Similarly, the perfect smile setup engine 2202 can transmit a request (block A, 150) to the tooth library selection engine 2210 for a subset of tooth libraries and/or at least one candidate tooth library to be used in designing veneers for a particular patient (or other dental appliances that may be used for improving the patient's smile, including but not limited to orthodontics). The engine 2210 can use different selection criteria for the perfect smile setup engine 2202 request versus the auto-design denture setup engine 2200 request. For example, the engine 2210 can compare aggregated tooth widths for tooth libraries with the patient's teeth. Designing veneers may have smaller degrees of freedom regarding movement and position than designing dentures because the veneers have to fit on an outer, facial, or front surface of the patient's teeth and without negatively impacting the patient's appearance. As another example, the auto-design implant setup engine 2206 can transmit a request (block A, 150) to the tooth library selection engine 2210 for a subset of tooth libraries and/or at least one candidate tooth library to be used in designing dental implants or inserts (e.g., crowns, caps) for a particular patient. The engine 2210 can use different selection criteria for the auto-design implant setup engine 2206 request versus the auto-design denture setup engine 2200 request and/or the perfect smile setup engine 2202 request. Designing implants and inserts may have smaller degrees of freedom regarding movement and position than designing dentures or even veneers because the implants or inserts have to fit into surface area of the patient's existing teeth and/or bone in the patient's upper and/or lower jaws. Refer to U.S. patent application Ser. No. 18/328,461 entitled “Systems and Methods for Library-Based Tooth Selection in Digital Dental Appliance Design,” filed on Jun. 2, 2023, the disclosure of which is incorporated by reference in its entirety.

FIG. 23 is an example architecture of a computing device 2950, which can be used to implement aspects according to the present disclosure. The computing device 2950 can be used to execute an operating system, application programs, and software modules to perform the denture design processes described herein. The computing device 2950 includes, in some embodiments, at least one processing device 2960, such as a central processing unit (CPU). Examples of computing devices suitable for the computing device 2950 include a desktop computer, a laptop computer, a tablet computer, a mobile computing device (such as a smartphone, an iPod® or iPad® mobile digital device, or other mobile devices), or other devices configured to process digital instructions. The system memory 2962 includes read only memory 2966 and random-access memory 2968. A basic input/output system 2970 containing the basic routines that act to transfer information within computing device 2950, such as during start up, is typically stored in the read only memory 2966.

The computing device 2950 also includes a secondary storage device 2972 in some embodiments, such as a hard disk drive, for storing digital data. The secondary storage device 2972 is connected to the system bus 2964 by a secondary storage interface 2974. The secondary storage devices 2972 and their associated computer readable media provide nonvolatile storage of computer readable instructions (including application programs and program modules), data structures, and other data for the computing device 2950. A number of program modules can be stored in secondary storage device 2972 or system memory 2962, including an operating system 2976, one or more application programs 2978, other program modules 2980 and program data 2982.

In some embodiments, a user provides inputs to the computing device 2950 through one or more input devices 2984. Examples of input devices 2984 include a keyboard 2986, mouse 2988, microphone 2990, and touch sensor 2992 (such as a touchpad or touch sensitive display). Other embodiments include other input devices 2984. The input devices are often connected to the processing device 2960 through an input/output interface 2994 that is coupled to the system bus 2964. These input devices 2984 can be connected by any number of input/output interfaces, such as a parallel port, serial port, game port, or a universal serial bus. Wireless communication between input devices and the interface 2994 is possible as well, and includes infrared, BLUETOOTH® wireless technology, 802.11a/b/g/n, cellular, ultra-wideband (UWB), ZigBee, or other radio frequency communication systems in some possible embodiments.

In this example embodiment, a display device 2996, such as a monitor, liquid crystal display device, projector, or touch sensitive display device, is also connected to the system bus 2964 via an interface, such as a video adapter 2998. In addition to the display device 2996, the computing device 2950 can include various other peripheral devices (not shown), such as speakers or a printer.

When used in a local area networking environment or a wide area networking environment (such as the Internet), the computing device 2950 is typically connected to the network through a network interface 3000, such as an Ethernet interface or WiFi interface. Other possible embodiments use other communication devices. For example, some embodiments of the computing device 2950 include a modem for communicating across the network.

The computing device 2950 typically includes at least some form of computer readable media. Computer readable media includes any available media that can be accessed by the computing device 2950. By way of example, computer readable media include computer readable storage media and computer readable communication media. Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 2950. Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device 2950 can also be an example of programmable electronics, which may include one or more such computing devices, and when multiple computing devices are included, such computing devices can be coupled together with a suitable data communication network so as to collectively perform the various functions, methods, or operations disclosed herein.

Although a few implementations have been described in detail above, other modifications are possible. Moreover, other mechanisms for performing the systems and methods described in this document may be used. In addition, the logic flows depicted in the figures do not require the particular order shown, or sequential order, to achieve desirable results. Other steps may be provided, or steps may be eliminated, from the described flows, and other components may be added to, or removed from, the described systems. Accordingly, other implementations are within the scope of the following claims.

What is claimed is:

1. A method for performing an auto-setup of a digital denture model, the method comprising:
accessing, by a computer system, a digital denture model for a patient, wherein the digital denture model comprises upper teeth and lower teeth;
defining, by the computer system, an arch form for the lower teeth in the digital denture model, wherein the arch form is aligned with (i) a buccal side of anterior teeth of the lower teeth and (ii) buccal cusps of posterior teeth of the lower teeth, wherein a same arch form is used for the upper teeth;
defining, by the computer system, an occlusal plane relative to the arch form for the digital denture model;
identifying, by the computer system, a plurality of datums for each tooth of the upper and lower teeth in the digital denture model;
leveling, by the computer system, each tooth of the upper and lower teeth in the digital denture model based on the respective plurality of datums being positioned relative to the occlusal plane;
snapping, by the computer system, each tooth of the upper and lower teeth in the digital denture model to the arch form;
until a threshold level of movement is achieved between each tooth and at least one of (i) an adjacent tooth and (ii) a tooth in vertical contact, iteratively:
adjusting, by the computer system, positioning of the tooth in the digital denture model to resolve interproximal (IP) contacts, and
adjusting, by the computer system, vertical positioning of the tooth in the digital denture model; and
returning, by the computer system, the digital denture model having the adjusted upper and lower teeth.

2. The method of claim 1, wherein leveling, by the computer system, each tooth of the upper and lower teeth based on the respective plurality of datums being positioned relative to the occlusal plane comprises: rotating the tooth around a line perpendicular through marginal ridge datums of the tooth.

3. The method of claim 1, wherein leveling, by the computer system, each tooth of the upper and lower teeth based on the respective plurality of datums being positioned relative to the occlusal plane comprises: torqueing the tooth using buccal and distal cusp tip datums of the tooth.

4. The method of claim 1, wherein leveling, by the computer system, each tooth of the upper and lower teeth based on the respective plurality of datums being positioned relative to the occlusal plane comprises: tipping the tooth mesially or distally using marginal ridge datums of the tooth.

5. The method of claim 1, wherein leveling, by the computer system, each tooth of the upper and lower teeth based on the respective plurality of datums being positioned relative to the occlusal plane comprises:
identifying a reference plane defined by the plurality of datums on an anterior tooth; and
tipping the anterior tooth according to the reference plane at a pivot point of the anterior tooth, wherein tipping the anterior tooth comprises leveling a tip of the anterior tooth with the occlusal plane.

6. The method of claim 1, wherein leveling, by the computer system, each tooth of the upper and lower teeth based on the respective plurality of datums being positioned relative to the occlusal plane comprises:
identifying a pivot point for a posterior tooth as a midpoint between 2 marginal ridge datums for the posterior tooth; and
rotating the posterior tooth around a line perpendicular to the midpoint to level the posterior tooth with the occlusal plane.

7. The method of claim 6, further comprising torqueing the posterior tooth using at least one of buccal cusp datums and distal cusp datums to cause a cusp of the posterior tooth to be parallel to the occlusal plane.

8. The method of claim 6, further comprising tipping the posterior tooth in at least one direction of mesially and distally using the 2 marginal ridge datums for the posterior tooth.

9. The method of claim 1, wherein snapping, by the computer system, each tooth of the upper and lower teeth to the arch form comprises:
for each tooth from a midline to a last molar in the upper teeth, snapping the tooth tangent to the arch form; and
for each tooth from the midline to a last molar in the lower teeth, snapping the tooth tangent to the arch form.

10. The method of claim 9, wherein for each tooth from a midline to a last molar in the upper teeth, snapping the tooth tangent to the arch form comprises:
snapping central incisors to the midline and tangent to the arch form;
snapping lateral teeth;
snapping canines so that respective cusp tips of the canines are positioned (i) relative to a tangent line on the arch form or (ii) a threshold distance outside of the arch form; and
for each molar and upper bicuspid tooth, (iii) rotating the tooth so that respective marginal ridge datums are tangent to the arch form and (iv) positioning the tooth buccal-lingually so that the marginal ridge datums are aligned on the arch form.

11. The method of claim 9, wherein for each tooth from the midline to a last molar in the lower teeth, snapping the tooth tangent to the arch form comprises:
snapping lower incisors to the midline and inside a tangent line so that the arch form touches a buccal side of the lower incisors;
snapping lower canines so that cusp tips of the lower canines are positioned (i) relative to the tangent line inside the arch form or (ii) a threshold distance inside of the arch form; and
for each molar and lower posterior tooth, translating the tooth lingually so that a respective buccal cusp tip is positioned on the arch form.

12. The method of claim 1, wherein adjusting, by the computer system, positioning of the tooth to resolve interproximal (IP) contacts comprises:
for each tooth, generating a bounding box;
for each tooth, identifying a center point of the tooth as a center point in the bounding box;
identifying a vector between center points of the teeth;
selecting the tooth at a defined position, the defined position being a midline; and
moving the tooth along the vector between the tooth and the adjacent tooth to (i) maintain relative orientation, remove overlap, and (ii) put the tooth in contact with the adjacent tooth at a predefined contact point.

13. The method of claim 12, further comprising: iteratively adjusting the vector between each next set of adjacent teeth and iteratively moving each next set of adjacent teeth along the respective vector until a last tooth is moved.

14. The method of claim 13, further comprising:
selecting a second tooth at a second defined position, the second defined position being a side of the midline that is opposite the defined position of the tooth; and
iteratively moving teeth adjacent the second tooth until a last tooth on the side of the midline that is opposite the defined position of the tooth is moved. The method of claim 1, wherein adjusting, by the computer system, vertical positioning of the tooth comprises moving each tooth of the lower teeth in a direction perpendicular to the occlusal plane until the tooth contacts the occlusal plane.

16. The method of claim 1, wherein adjusting, by the computer system, vertical positioning of the tooth comprises socking each tooth of the upper teeth until the tooth contacts one or more of the lower teeth.

17. The method of claim 1, wherein adjusting, by the computer system, vertical positioning of the tooth comprises:
identifying a center point between 3 adjacent teeth to define a buccal vector as perpendicular to the center point;
for each tooth, adjusting the tooth buccally, lingually, and down based on the buccal vector;
measuring a distance between the adjusted tooth and at least one tooth vertically in contact with the adjusted tooth;
determining whether the distance is within a predetermined threshold distance;
reducing the distance in half based on determining that the distance is not within the predetermined threshold distance;
moving the adjusted tooth in an opposite direction of the adjustments by the reduced distance;
measuring a new distance between the adjusted tooth and the at least one tooth vertically in contact with the adjusted tooth; and
iteratively moving the adjusted tooth buccally, lingually, down, and up until the measured distance is within the predetermined threshold distance.

18. The method of claim 1, further comprising:
receiving, by the computer system, patient tooth data, wherein the patient tooth data comprises at least one image of teeth of the patient;
selecting, by the computer system and from a data store, a candidate tooth library from amongst a plurality of static tooth libraries based at least in part on the patient tooth data; and
generating, by the computer system, the digital denture model based on the patient tooth data and the candidate tooth library, wherein generating the digital denture model comprises overlaying teeth of the candidate tooth library over corresponding teeth of the digital denture model.

19. The method of claim 1, further comprising:
transmitting the digital denture model to a user device for presentation in a graphical user interface (GUI) at the user device;
receiving, by the computer system and from the user device, user input indicating one or more adjustments to at least one tooth of the upper teeth and the lower teeth in the digital denture model;
and iteratively performing, by the computer system and based on the user input, at least one of:
(i) leveling the at least one tooth,
(ii) snapping the at least one tooth to the arch form, and
(iii) until a threshold level of movement is achieved between the at least one tooth and at least one of (a) an adjacent tooth and (b) a tooth in vertical contact, adjusting a position of the at least one tooth to resolve IP contacts and adjusting a vertical positioning of the at least one tooth.

20. The method of claim 1, wherein snapping, by the computer system, each tooth of the upper and lower teeth to the arch form comprises aligning the tooth to the arch form using an iterative fitting algorithm, the iterative fitting algorithm being an iterative closest point algorithm.
